(19) United States
(12) Patent Application Publication
(10) Pub. No.: US 2023/0397891 A1
(43) Pub. Date: Dec. 14, 2023

(54) EAR-WEARABLE DEVICES FOR DETECTING, MONITORING, OR PREVENTING HEAD INJURIES

(71) Applicant: Starkey Laboratories, Inc., Eden Prairie, MN (US)

(72) Inventors: Babak Talebanpour, Bloomington, MN (US); Arghavan Talebanpour, Eden Prairie, MN (US); Yoshi Kasahara, St. Louis Park, MN (US); Gregory John Haubrich, Champlin, MN (US)

(21) Appl. No.: 18/033,937

(22) PCT Filed: Oct. 28, 2021
(86) PCT No.: PCT/US2021/057064

§ 371 Date: Apr. 26, 2023

Related U.S. Application Data
(60) Provisional application No. 63/107,991, filed on Oct. 30, 2020.

(51) Int. Cl. A61B 5/00; A61B 5/024; A61B 5/11; H04R 1/10; A61B 5/021

(52) U.S. Cl. CPC A61B 5/7275; A61B 5/6817; A61B 5/746; A61B 5/024; A61B 5/7282; A61B 5/1122; H04R 1/1016; A61B 5/021

(57) ABSTRACT

Embodiments herein relate to ear-wearable devices that can be used for detecting, monitoring, and/or preventing head injuries. In an embodiment, an ear-wearable device is included having a control circuit, a microphone, a motion sensor, and a power supply circuit, wherein the ear-wearable device is configured to monitor signals from the motion sensor to detect an occurrence of head movement consistent with possible head trauma. In an embodiment, a method of tracking head trauma with an ear-wearable device is included. The method can include evaluating signals from a motion sensor that is part of the ear-wearable device, calculating at least one of peak rotational or linear acceleration and/or peak rotational velocity, and comparing calculated peak rotational or linear acceleration and/or peak rotational velocity against one or more threshold values for head trauma. Other embodiments are also included herein.

This application is being filed as a PCT International Patent application on Oct. 28, 2021 in the name of Starkey Laboratories, Inc., a U.S. national corporation, applicant for the designation of all countries, and Babak Talebanpour, a U.S. Citizen, and Arghavan Talebanpour, a U.S. Citizen, and Yoshi Kasahara, a U.S. Citizen, and Gregory John Haubrich, a U.S. Citizen, inventor(s) for the designation of all countries, and claims priority to U.S. Provisional Patent Application No. 63/107,991, filed Oct. 30, 2020, the contents of which are herein incorporated by reference in its entirety.

### FIELD

Embodiments herein relate to ear-wearable devices. More specifically, embodiments herein relate to ear-wearable devices that can be used for detecting, monitoring, and/or preventing head injuries.

### BACKGROUND

Traumatic brain injury (TBI) is a significant cause of mortality and morbidity worldwide. It is estimated that 1.7 million people sustain a TBI annually resulting a high number of hospitalizations and deaths.

The risks of sustaining a TBI particularly for younger age groups while participating in sports is now widely appreciated. However, the risks of sustaining a TBI for the elderly, such as resulting from a fall, is not as widely recognized. Yet, the TBIs suffered by the elderly make up a significant proportion of the total number of TBIs sustained annually. Thus, the elderly represent a significantly underserved population that is at high risk for head injuries.

Head injuries such as TBIs and, particularly, the extent and severity of the brain injury, often go undiagnosed until long after the damage has occurred and even after the damage is compounded by further injuries/concussions. In the elderly, the effects of TBIs may be masked by or confused with symptoms of other conditions. This makes it very difficult for clinicians and care providers to accurately diagnose and treat TBIs and/or provide for preventative measures to prevent additional injuries

In addition, repeated head impacts that may not rise to the level of diagnosed concussions may still lead to other conditions such chronic traumatic encephalopathy (CTE). However, the cumulative volume of such sub-concussive head impacts can be very difficult to track over time.

### SUMMARY

Embodiments herein relate to ear-wearable devices that can be used for detecting, monitoring, and/or preventing head injuries. In a first aspect, an ear-wearable device is included having a control circuit, a microphone, a motion sensor, and a power supply circuit, wherein the ear-wearable device is configured to monitor signals from the motion sensor to detect an occurrence of head movement consistent with possible head trauma.

In a second aspect, in addition to one or more of the preceding or following aspects, or in the alternative to some aspects, the ear-wearable device is configured to record detected occurrences of head movement consistent with possible head trauma.

In a third aspect, in addition to one or more of the preceding or following aspects, or in the alternative to some aspects, signals from the motion sensor that cross a threshold value of acceleration and/or velocity are deemed to be consistent with possible head trauma.

In a fourth aspect, in addition to one or more of the preceding or following aspects, or in the alternative to some aspects, the ear-wearable device is configured to calculate a cumulative impact index based on the recorded detected occurrences of head movement consistent with possible head trauma.

In a fifth aspect, in addition to one or more of the preceding or following aspects, or in the alternative to some aspects, the ear-wearable device is configured to change a sampling rate of the motion sensor if movement is detected consistent with an initial phase of an impact event.

In a sixth aspect, in addition to one or more of the preceding or following aspects, or in the alternative to some aspects, the ear-wearable device is configured to increase a sampling rate of the motion sensor if movement is detected consistent with an initial phase of an impact event.

In a seventh aspect, in addition to one or more of the preceding or following aspects, or in the alternative to some aspects, the ear-wearable device is configured monitor signals from the motion sensor to detect at least one of dizziness or a fall after detecting an occurrence of head movement consistent with possible head trauma.

In an eighth aspect, in addition to one or more of the preceding or following aspects, or in the alternative to some aspects, the ear-wearable device is configured to generate an audio warning when an occurrence of head movement consistent with possible head trauma is detected.

In a ninth aspect, in addition to one or more of the preceding or following aspects, or in the alternative to some aspects, the ear-wearable device is configured to generate an audio warning and send the same to a responsible third party when an occurrence of head movement consistent with possible head trauma is detected.

In a tenth aspect, in addition to one or more of the preceding or following aspects, or in the alternative to some aspects, the ear-wearable device is configured to generate a query for a wearer of the ear-wearable device when an occurrence of head movement consistent with possible head trauma is detected.

In an eleventh aspect, in addition to one or more of the preceding or following aspects, or in the alternative to some aspects, the ear-wearable device is configured to generate a delayed query for a wearer of the ear-wearable device when an occurrence of head movement consistent with possible head trauma is detected.

In a twelfth aspect, in addition to one or more of the preceding or following aspects, or in the alternative to some aspects, the ear-wearable device in configured to process signals from the motion sensor to measure at least one of linear and rotational acceleration and/or rotational velocity.

In a thirteenth aspect, in addition to one or more of the preceding or following aspects, or in the alternative to some aspects, the ear-wearable device in configured to process signals from the motion sensor to estimate linear acceleration at a center of gravity of the head of a wearer of the ear-wearable device.

In a fourteenth aspect, in addition to one or more of the preceding or following aspects, or in the alternative to some aspects, the ear-wearable device is configured to monitor signals from the microphone to detect a possible impact event.

In a fifteenth aspect, in addition to one or more of the preceding or following aspects, or in the alternative to some aspects, the ear-wearable device is configured to receive signals regarding detected movement from an accessory device.

In a sixteenth aspect, in addition to one or more of the preceding or following aspects, or in the alternative to some aspects, the accessory device includes at least one of a second ear-wearable device, a smart phone, and a secondary wearable device.

In a seventeenth aspect, in addition to one or more of the preceding or following aspects, or in the alternative to some aspects, the ear-wearable device is configured to estimate a degree of possible damage to a neck of the ear-wearable device wearer based on a comparison of signals from the motion sensor and signals from the accessory device.

In an eighteenth aspect, in addition to one or more of the preceding or following aspects, or in the alternative to some aspects, the ear-wearable device is configured to a generate an alert if an occurrence of possible head trauma is detected that crosses a threshold value.

In a nineteenth aspect, in addition to one or more of the preceding or following aspects, or in the alternative to some aspects, the ear-wearable device is configured to select an appropriate first target recipient for the alert.

In a twentieth aspect, in addition to one or more of the preceding or following aspects, or in the alternative to some aspects, the ear-wearable device is configured to select an appropriate first target recipient for the alert based on a predicted severity of possible head trauma.

In a twenty-first aspect, in addition to one or more of the preceding or following aspects, or in the alternative to some aspects, the predicted severity is based on factors including one or more of peak detected acceleration, peak detected velocity, post-incident monitoring data, pre-incident monitoring data, and initial phase detection.

In a twenty-second aspect, in addition to one or more of the preceding or following aspects, or in the alternative to some aspects, wherein post-incident monitoring data includes at least one of microphone signals and motion sensor signals.

In a twenty-third aspect, in addition to one or more of the preceding or following aspects, or in the alternative to some aspects, the device can further include a physiological sensor, the physiological sensor can include at least one selected from the group consisting of a blood pressure sensor and a heart rate sensor.

In a twenty-fourth aspect, in addition to one or more of the preceding or following aspects, or in the alternative to some aspects, the ear-wearable device is configured to stream data based on the signals from at least one of the microphone and the motion sensor after detection an occurrence of head movement consistent with possible head trauma.

In a twenty-fifth aspect, an ear-wearable device system is included having a first ear-wearable device including a first control circuit, a first microphone, a first motion sensor, and a first power supply circuit and a second ear-wearable device including a second control circuit, a second microphone, a second motion sensor, and a second power supply circuit. The ear-wearable device system can be configured to process signals from the first motion sensor and the second motion sensor to calculate at least one of linear and rotational acceleration and/or rotational velocity and can be configured to monitor at least one of the linear or rotational acceleration and/or rotational velocity to detect an occurrence of peak acceleration and/or velocity consistent with possible head trauma.

In a twenty-sixth aspect, in addition to one or more of the preceding or following aspects, or in the alternative to some aspects, the ear-wearable device system is configured to change a sampling rate of at least one of the first motion sensor and the second motion sensor if movement is detected consistent with an initial phase of an impact event.

In a twenty-seventh aspect, in addition to one or more of the preceding or following aspects, or in the alternative to some aspects, the ear-wearable device system is configured to increase a sampling rate of at least one of the first motion sensor and the second motion sensor if movement is detected consistent with an initial phase of an impact event.

In a twenty-eighth aspect, in addition to one or more of the preceding or following aspects, or in the alternative to some aspects, the ear-wearable device system in configured to process signals from at least one of the first motion sensor and the second motion sensor to estimate linear acceleration and/or rotational acceleration or velocity at a center of gravity of the head of a wearer of the ear-wearable device system.

In a twenty-ninth aspect, in addition to one or more of the preceding or following aspects, or in the alternative to some aspects, the ear-wearable device system is configured to record detected occurrences of peak acceleration and/or velocity consistent with possible head trauma.

In a thirtieth aspect, in addition to one or more of the preceding or following aspects, or in the alternative to some aspects, the ear-wearable device system is configured to calculate a cumulative impact index based on the recorded detected occurrences of peak acceleration and/or velocity consistent with possible head trauma.

In a thirty-first aspect, in addition to one or more of the preceding or following aspects, or in the alternative to some aspects, the ear-wearable device system is configured monitor signals from at least one of the first motion sensor and the second motion sensor to detect at least one of dizziness or a fall after detecting an occurrence of head movement consistent with possible head trauma.

In a thirty-second aspect, in addition to one or more of the preceding or following aspects, or in the alternative to some aspects, the ear-wearable device system is configured to generate an audio warning when an occurrence of peak acceleration and/or velocity consistent with possible head trauma is detected.

In a thirty-third aspect, in addition to one or more of the preceding or following aspects, or in the alternative to some aspects, the ear-wearable device system is configured to generate an audio warning and send the same to a responsible third party when an occurrence of peak acceleration and/or velocity consistent with possible head trauma is detected.

In a thirty-fourth aspect, in addition to one or more of the preceding or following aspects, or in the alternative to some aspects, the ear-wearable device system is configured to generate a query for a wearer of the ear-wearable device system when an occurrence of peak acceleration and/or velocity consistent with possible head trauma is detected.

In a thirty-fifth aspect, in addition to one or more of the preceding or following aspects, or in the alternative to some aspects, the ear-wearable device system is configured to generate a delayed query for a wearer of the ear-wearable device system when an occurrence of peak acceleration and/or velocity consistent with possible head trauma is detected.

In a thirty-sixth aspect, in addition to one or more of the preceding or following aspects, or in the alternative to some aspects, the ear-wearable device system is configured to monitor signals from at least one of the first microphone and the second microphone to detect a possible impact event.

In a thirty-seventh aspect, in addition to one or more of the preceding or following aspects, or in the alternative to some aspects, the ear-wearable device system is configured to receive signals regarding detected movement from an accessory device.

In a thirty-eighth aspect, in addition to one or more of the preceding or following aspects, or in the alternative to some aspects, the accessory device includes at least one of a smart phone and a secondary wearable device.

In a thirty-ninth aspect, in addition to one or more of the preceding or following aspects, or in the alternative to some aspects, the ear-wearable device system is configured to estimate a degree of possible damage to a neck of the ear-wearable device system wearer based on a comparison of signals from at least one of the first motion sensor and the second motion sensor and signals from the accessory device.

In a fortieth aspect, in addition to one or more of the preceding or following aspects, or in the alternative to some aspects, the ear-wearable device system is configured to a generate an alert if an occurrence of possible head trauma is detected that crosses a threshold value.

In a forty-first aspect, in addition to one or more of the preceding or following aspects, or in the alternative to some aspects, the ear-wearable device system is configured to select an appropriate first target recipient for the alert.

In a forty-second aspect, in addition to one or more of the preceding or following aspects, or in the alternative to some aspects, the ear-wearable device system is configured to select an appropriate first target recipient for the alert based on a predicted severity of possible head trauma.

In a forty-third aspect, in addition to one or more of the preceding or following aspects, or in the alternative to some aspects, the predicted severity is based on factors including one or more of peak detected acceleration, peak detected velocity, post-incident monitoring data, pre-incident monitoring data, and initial phase detection.

In a forty-fourth aspect, in addition to one or more of the preceding or following aspects, or in the alternative to some aspects, wherein post-incident monitoring data includes at least one of microphone signals and motion sensor signals.

In a forty-fifth aspect, in addition to one or more of the preceding or following aspects, or in the alternative to some aspects, devices of the system can further include a physiological sensor, the physiological sensor can include at least one selected from the group consisting of a blood pressure sensor and a heart rate sensor.

In a forty-sixth aspect, in addition to one or more of the preceding or following aspects, or in the alternative to some aspects, the ear-wearable device system is configured to stream data based on the signals from at least one of the first microphone, the first motion sensor, the second microphone, and the second motion sensor after detection an occurrence of head movement consistent with possible head trauma.

In a forty-seventh aspect, in addition to one or more of the preceding or following aspects, or in the alternative to some aspects, wherein signals from the first motion sensor or the second motion sensor that cross a threshold value of acceleration and/or velocity are consistent with possible head trauma.

In a forty-eighth aspect, a method of tracking head trauma with an ear-wearable device is included, the method including evaluating signals from a motion sensor that is part of the ear-wearable device, calculating at least one of peak rotational or linear acceleration and/or rotational velocity, and comparing calculated peak rotational or linear acceleration and/or rotational velocity against one or more threshold values for head trauma.

In a forty-ninth aspect, in addition to one or more of the preceding or following aspects, or in the alternative to some aspects, the method can further include recording calculated peak acceleration and/or velocity.

In a fiftieth aspect, in addition to one or more of the preceding or following aspects, or in the alternative to some aspects, wherein recording calculated peak acceleration and/or velocity further includes calculating a cumulative impact index based on the recorded peak acceleration and/or velocity values.

In a fifty-first aspect, in addition to one or more of the preceding or following aspects, or in the alternative to some aspects, the method can further include changing a sampling rate of the motion sensor if movement is detected consistent with an initial phase of an impact event.

In a fifty-second aspect, in addition to one or more of the preceding or following aspects, or in the alternative to some aspects, the method can further include increasing a sampling rate of the motion sensor if movement is detected consistent with an initial phase of an impact event.

In a fifty-third aspect, in addition to one or more of the preceding or following aspects, or in the alternative to some aspects, the method can further include monitoring signals from the motion sensor to detect at least one of dizziness or a fall after detecting an occurrence of peak acceleration and/or velocity consistent with possible head trauma.

In a fifty-fourth aspect, in addition to one or more of the preceding or following aspects, or in the alternative to some aspects, the method can further include generating an audio warning when an occurrence of peak acceleration and/or velocity consistent with possible head trauma is detected.

In a fifty-fifth aspect, in addition to one or more of the preceding or following aspects, or in the alternative to some aspects, the method can further include generating an audio warning and sending the same to a responsible third party when an occurrence of peak acceleration and/or velocity consistent with possible head trauma is detected.

In a fifty-sixth aspect, in addition to one or more of the preceding or following aspects, or in the alternative to some aspects, the method can further include generating a query for a wearer of the ear-wearable device when an occurrence of peak acceleration and/or velocity consistent with possible head trauma is detected.

In a fifty-seventh aspect, in addition to one or more of the preceding or following aspects, or in the alternative to some aspects, the method can further include generating a delayed query for a wearer of the ear-wearable device when an occurrence of peak acceleration and/or velocity consistent with possible head trauma is detected.

In a fifty-eighth aspect, in addition to one or more of the preceding or following aspects, or in the alternative to some aspects, the method can further include estimating linear acceleration and/or velocity at a center of gravity of the head of a wearer of the ear-wearable device.

In a fifty-ninth aspect, in addition to one or more of the preceding or following aspects, or in the alternative to some aspects, the method can further include evaluating signals from a microphone that is part of the ear-wearable device to detect a possible impact event.

In a sixtieth aspect, in addition to one or more of the preceding or following aspects, or in the alternative to some aspects, the method can further include receiving signals regarding detected motion from an accessory device.

In a sixty-first aspect, in addition to one or more of the preceding or following aspects, or in the alternative to some aspects, receiving signals regarding detected motion from an accessory device further includes estimating a degree of possible damage to a neck of the ear-wearable device wearer based on a comparison of signals from the motion sensor and signals from the accessory device.

In a sixty-second aspect, in addition to one or more of the preceding or following aspects, or in the alternative to some aspects, the method can further include generating an alert if an occurrence of possible head trauma is detected that crosses a threshold value.

In a sixty-third aspect, in addition to one or more of the preceding or following aspects, or in the alternative to some aspects, the method can further include selecting an appropriate first target recipient for the alert.

In a sixty-fourth aspect, in addition to one or more of the preceding or following aspects, or in the alternative to some aspects, the method can further include selecting an appropriate first target recipient for the alert based on a predicted severity of possible head trauma.

In a sixty-fifth aspect, in addition to one or more of the preceding or following aspects, or in the alternative to some aspects, the method can further include predicting severity based on factors including one or more of peak detected acceleration, peak detected velocity, post-incident monitoring data, pre-incident monitoring data, and initial phase detection.

In a sixty-sixth aspect, in addition to one or more of the preceding or following aspects, or in the alternative to some aspects, wherein post-incident monitoring data includes at least one of microphone signals and motion sensor signals.

In a sixty-seventh aspect, in addition to one or more of the preceding or following aspects, or in the alternative to some aspects, the method can further include evaluating sensor data from at least one sensor selected from the group consisting of a blood pressure sensor and a heart rate sensor.

In a sixty-eighth aspect, in addition to one or more of the preceding or following aspects, or in the alternative to some aspects, the method can further include streaming data based on signals from at least one of the motion sensor and a microphone after detection an occurrence of head movement consistent with possible head trauma.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of an ear-wearable device wearer in accordance with various embodiments herein.

FIG. 2 is a schematic view of an ear-wearable device being worn in accordance with various embodiments herein.

FIG. 3 is a schematic view of an ear-wearable device in accordance with various embodiments herein.

FIG. 4 is a schematic view of an ear-wearable device within an ear in accordance with various embodiments herein.

FIG. 5 is a front elevation view of ear-wearable devices being worn in accordance with various embodiments herein.

FIG. 6 is a side elevation view of ear-wearable devices being worn in accordance with various embodiments herein.

FIG. 7 is a top plan view of ear-wearable devices being worn in accordance with various embodiments herein.

FIG. 8 is a schematic view of an ear-wearable device being worn in accordance with various embodiments herein.

FIG. 9 is a schematic view of an ear-wearable device being worn by a driver of a vehicle in accordance with various embodiments herein.

FIG. 10 is a schematic view of some components of an ear-wearable device system in accordance with various embodiments herein.

FIG. 11 is a schematic view of an accessory device in accordance with various embodiments herein.

FIG. 12 is a schematic view of an ear-wearable device wearer falling in accordance with various embodiments herein.

FIG. 13 is a flow chart of operations of a method in accordance with various embodiments herein.

FIG. 14 is a schematic view of components of an ear-wearable device in accordance with various embodiments herein.

FIG. 15 is a schematic view of components of an accessory device in accordance with various embodiments herein.

FIG. 16 is a schematic view of an in-the-ear style custom ear-wearable device in accordance with various embodiments herein

FIG. 17 is a schematic view of an ear-wearable device disposed within the ear of a wearer in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Embodiments herein enable measurements of impact to the head and neck by measuring velocities and/or accelerations. Embodiments herein can be used to actively assess the severity of the head, or neck injuries associated with impacts to the head or head/body velocities and/or acceleration.

In various embodiments herein, devices and/or systems can maintain a cumulative impact index in order to account for all detected head trauma (including one or more of head impacts generally, sub-concussive head impacts, and concussive head impacts) to provide for a more accurate view of the total amount (e.g., total volume) of head trauma experienced by an individual.

In addition, embodiments herein can be worn on or in the ear and thus enable highly accurate measurements of linear and/or rotational velocities and/or accelerations without the need for helmets or other head wearable devices.

In some embodiments herein, devices and/or systems can initiate appropriate action based on an estimated severity of head trauma. For example, in some cases, the device and/or system can notify a care provider or loved one regarding a detected incident of head trauma. In other cases, such when more severe head trauma is detected, the device and/or system can notify an emergency responder.

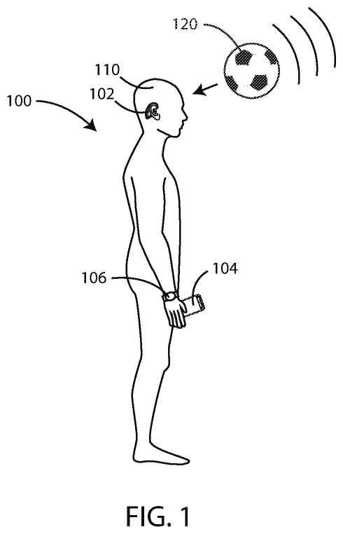

Referring now to FIG. 1, a schematic view of an ear-wearable device wearer 100 is shown in accordance with various embodiments herein. The device wearer 100 is wearing an ear-wearable device 102. In this view, the device wearer 100 also has an accessory device 104 and a secondary wearable device 106.

In this view, a ball 120 is about to strike the head 110 of the device wearer 100. As described herein, the ear-wearable device 102 can be configured to monitor signals from the motion sensor to detect an occurrence of head 110 movement consistent with possible head 110 trauma, such as could occur upon impact with the ball 120. Details of head movement consistent with head trauma are described in greater detail below, however, in various embodiments, signals from the motion sensor that cross a threshold value of acceleration (such as peak acceleration) and/or velocity (such as peak velocity) are consistent with possible head 110 trauma.

In various embodiments, the ear-wearable device 102 can be configured to record detected occurrences of head 110 movement consistent with possible head 110 trauma. In various embodiments, the ear-wearable device 102 is configured to calculate a cumulative impact index based on the recorded detected occurrences of head 110 movement consistent with possible head 110 trauma

Various alerts and/or warnings can be generated by embodiments of ear-wearable devices herein. Alerts or warnings can be audio, visual (such as through an accessory device), haptic, or the like. In some embodiments, the ear-wearable device 102 can be configured to generate an audio warning when an occurrence of head 110 movement consistent with possible head 110 trauma is detected. In various embodiments, the ear-wearable device 102 can be configured to generate an audio warning and send the same to a responsible third party (which could be a care provider, a clinician, a coach, or the like) when an occurrence of head 110 movement consistent with possible head 110 trauma is detected. In various embodiments, the ear-wearable device 102 can be configured to a generate an alert if an occurrence of possible head 110 trauma is detected that crosses a threshold value. In various embodiments, the ear-wearable device 102 is configured to select an appropriate first target recipient for an alert. In various embodiments, the ear-wearable device 102 is configured to select an appropriate first target recipient for an alert based on a predicted severity of possible head 110 trauma. In various embodiments, the predicted severity can be based on factors including one or more of peak detected acceleration, peak detected velocity, post-incident monitoring data, pre-incident monitoring data, and initial phase detection data.

In various embodiments, the ear-wearable device can try to assess the condition of the device wearer 100 after the occurrence of possible head trauma. For example, in various embodiments, the ear-wearable device 102 can be configured to generate a query for a wearer of the ear-wearable device 102 when an occurrence of head 110 movement consistent with possible head trauma is detected. Exemplary queries are described in greater detail below but can relate to testing of the device wearer for their cognitive function including confusion and memory issues, asking them about their current condition, or the like. In various embodiments, the ear-wearable device 102 can be configured to generate a delayed query for a wearer of the ear-wearable device 102 when an occurrence of head 110 movement consistent with possible head trauma is detected. A delayed query can be akin to a follow-up assessment of the effects of the possible head trauma. Delayed queries can be similar to immediate queries or different. In some embodiments, delayed queries can relate to concentration and/or memory issues, irritability or other personality changes, sensitivity to light and/or noise, changes in sleep habits and/or sleep disturbances, psychological adjustment issues and/or depression, disorder of taste and smell, and the like.

In various embodiments, post-incident monitoring data includes at least one of microphone signals and motion sensor signals. As such, the ear-wearable device 102 can be configured monitor signals from the motion sensor to detect at least one of consciousness, dizziness, or a fall occurring after detecting an occurrence of head 110 movement consistent with possible head 110 trauma. In various embodiments, the ear-wearable device 102 can be configured to stream data based on the signals from at least one of a microphone and a motion sensor after detection an occurrence of head 110 movement consistent with possible head 110 trauma.

Detection of circumstances that may precede an impact event, even if the timing of the detection is not sufficient to warn the device wearer, can still be useful as it can allow the ear-wearable device to take steps to enhance detection and/or assessment of possible head trauma. For example, the sampling rate of various sensors can be increased to provide a richer data set to more accurately detect peak acceleration and/or peak velocity. In particular, peak acceleration and/or peak velocity values may occur within the first 5 to 10 milliseconds after head impact and capturing such peak values accurately requires high sampling rates. The sampling rate can be dynamically/adaptively adjusted to minimize unnecessary data and/or current drain. As another example, additional sensors can be turned on to produce a richer data set to more accurately detect possible head trauma. In some cases, an impact event may be preceded by unique sounds. For example, an auto accident may be preceded by the sounds of squealing tires. In various embodiments, the ear-wearable device 102 can be configured to monitor signals from a microphone to detect circumstances preceding a possible impact event (described further below). In some cases, an impact event may be preceded by unique movement of the device wearer. For example, a fall of the device wearer may precede a head impact event. As another example, a detected gait or movement pattern of the device wearer (such as one that is different than normal or erratic) may precede a head impact event. As another example, movement consistent with a seizure may precede a head impact event. As such, in various embodiments, the ear-wearable device can be configured to monitor signals from a motion sensor in order to detect circumstances preceding a possible impact event.

In some scenarios, the ear-wearable device or system can be configured to only rely on signals from sensors that are a part of the ear-wearable device or system. However, in various embodiments, the ear-wearable device 102 can be configured to receive signals regarding detected movement from an accessory device 104. In various embodiments, the accessory device 104 can include at least one of a second ear-wearable device, a smart phone, and/or a secondary wearable device.

In some cases, an accessory device 104 can pick up motion that is associated with another part of an individual's body other than the head. This can be useful to analyze as it can provide a more complete picture of the possible damage associated with the impact event. For example, in various embodiments, the ear-wearable device 102 is configured to estimate a degree of possible damage to a neck of the ear-wearable device 102 wearer based on a comparison of signals from a motion sensor and signals from an accessory device 104.

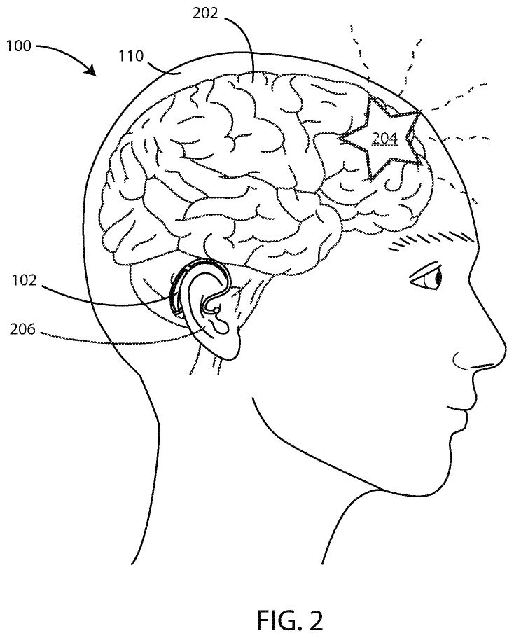

Referring now to FIG. 2, a schematic view of an ear-wearable device 102 being worn by a device wearer 100 is shown in accordance with various embodiments herein. Specifically, the ear-wearable device 102 is shown being worn on or in an ear 206 of the device wearer 100. FIG. 2 shows the brain 202 of the device wearer 100 inside of their head 110. An injury 204 to the brain 202 is schematically depicted. The injury 204 can be representative of any degree of head trauma from a severe concussion to a mild, sub-concussive blow.

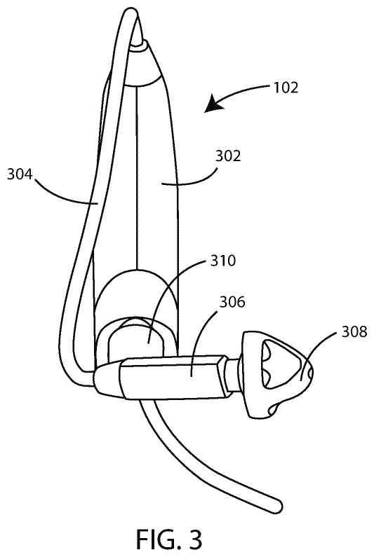

Referring now to FIG. 3, a schematic view of an ear-wearable device 102 is shown in accordance with various embodiments herein. The ear-wearable device 102 can include a hearing device housing 302. The hearing device housing 302 can define a battery compartment 310 into which a battery can be disposed to provide power to the device. The ear-wearable device 102 can also include a receiver 306 adjacent to an earbud 308. The receiver 306 can include a component that converts electrical impulses into sound, such as an electroacoustic transducer, speaker, or loudspeaker. Such components can be used to generate an audible stimulus in various embodiments herein. A cable 304 or connecting wire can include one or more electrical conductors and provide electrical communication between components inside of the hearing device housing 302 and components inside of the receiver 306.

The ear-wearable device 102 shown in FIG. 3 is a receiver-in-canal type device and thus the receiver is designed to be placed within the ear canal. However, it will be appreciated that many different form factors for ear-wearable devices are contemplated herein. As such, ear-wearable devices herein can include, but are not limited to, behind-the-ear (BTE), in-the ear (ITE), in-the-canal (ITC), invisible-in-canal (ITC), receiver-in-canal (RIC), receiver in-the-ear (RITE) and completely-in-the-canal (CIC) type hearing assistance devices.

The term “ear-wearable device” shall also refer to devices that can produce optimized or processed sound for persons with normal hearing. Ear-wearable devices herein can include hearing assistance devices. In some embodiments, the ear-wearable device can be a hearing aid falling under 21 C.F.R. § 801.420. In another example, the ear-wearable device can include one or more Personal Sound Amplification Products (PSAPs). In another example, the ear-wearable device can include one or more cochlear implants, cochlear implant magnets, cochlear implant transducers, and cochlear implant processors. In another example, the ear-wearable device can include one or more “hearable” devices that provide various types of functionality. In other examples, ear-wearable devices can include other types of devices that are wearable in, on, or in the vicinity of the user's ears. In other examples, ear-wearable devices can include other types of devices that are implanted or otherwise osseointegrated with the user's skull; wherein the device is able to facilitate stimulation of the wearer's ears via the bone conduction pathway.

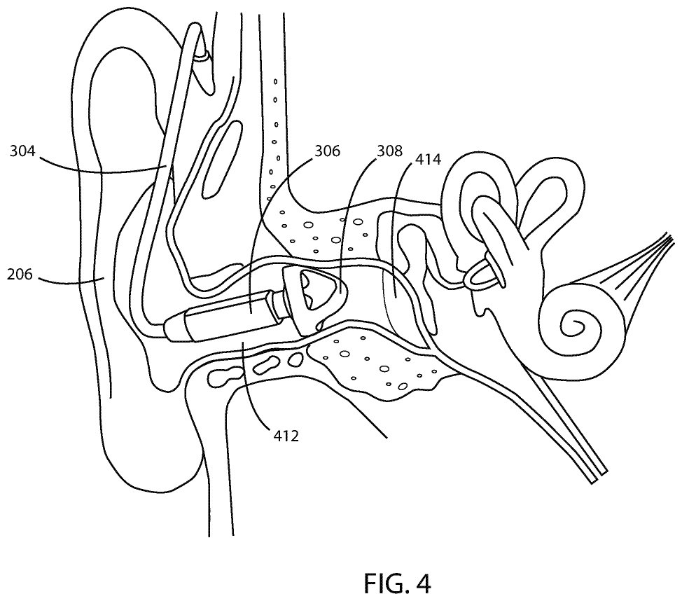

As mentioned above, the ear-wearable device 102 shown in FIG. 3 can be a receiver-in-canal type device and thus the receiver is designed to be placed within the ear canal. Referring now to FIG. 4, a schematic view is shown of an ear-wearable device 102 disposed within the ear of a subject in accordance with various embodiments herein. In this view, the receiver 306 and the earbud 308 are both within the ear canal 412, but do not directly contact the tympanic membrane 414. The hearing device housing is mostly obscured in this view behind the pinna 206, but it can be seen that the cable 304 passes over the top of the pinna 206 and down to the entrance to the ear canal 412.

In some embodiments, an ear-wearable device herein can be part of a system that may also include other components/devices. By way of example, in some embodiments, a system herein can also include an accessory device. The accessory device can communicate with the ear-wearable device(s) and exchange general data, sensor data, notifications, convey messages or commands, etc. In some embodiments, processing intensive tasks can be offloaded to the accessory device.

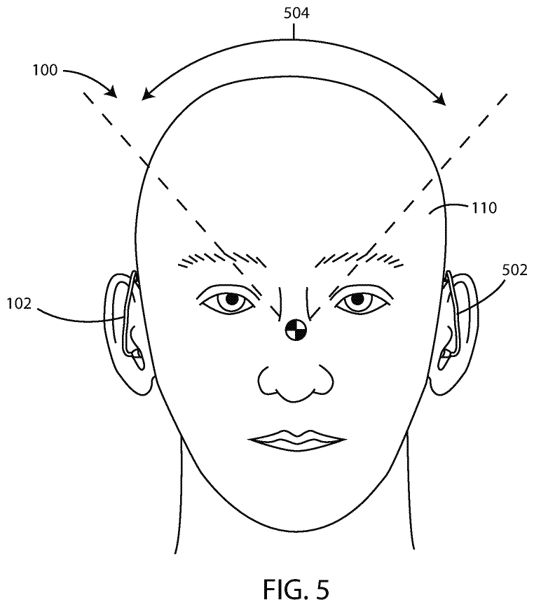

While not intending to be bound by theory, it is believed that a system herein including two ear-wearable devices can provide even better and more accurate information regarding occurrences of head trauma than can a single ear-wearable device. Data from a second ear-wearable device can also be used to provide redundancy and reliability since it is less likely both ear-wearable devices will become dislodged from the ears of the wearer versus a single ear-wearable device becoming dislodged. In some embodiments, in order to preserve battery life, the system can duty cycle between sensors associated with the two ear-wearable devices. Referring now to FIG. 5, a front elevation view of a pair of ear-wearable devices being worn is shown in accordance with various embodiments herein. In this view, the device wearer 100 is shown wearing an ear-wearable device 102 and a second ear-wearable device 502 on their head 110 (and specifically, on or in their ears). FIG. 5 also shows frontal (or coronal) plane rotational acceleration and/or velocity 504 being measured by the ear-wearable device 102.

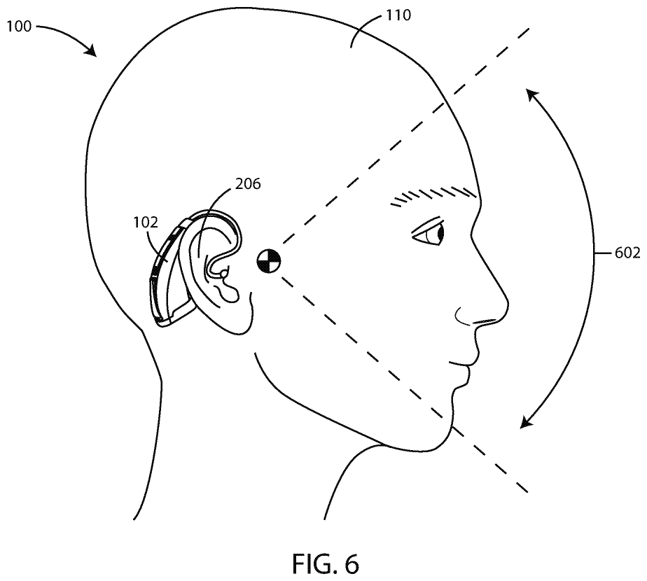

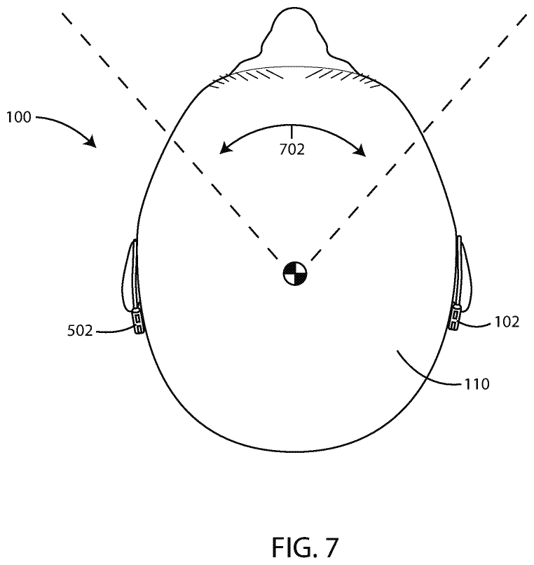

Referring now to FIG. 6, a side elevation view of ear-wearable devices being worn is shown in accordance with various embodiments herein. In this view, the head 110 is shown of the device wearer 100 along with an ear-wearable device 102 on their ear 206. It will be appreciated that the acceleration and/or velocity that can be used herein can take on various forms. In various embodiments, the ear-wearable device 102 in configured to process signals from the motion sensor to measure at least one of linear and rotational acceleration and/or rotational velocity. In particular, FIG. 6 shows sagittal or vertical plane rotational acceleration and/or velocity 602 being measured by the ear-wearable device 102. FIG. 7 is generally similar to FIG. 6 but shows a top plan view. FIG. 7 also shows the device wearer 100 wearing a second ear-wearable device 502. FIG. 7 shows an ear-wearable device 102. FIG. 7 shows a transverse or horizontal plane rotational acceleration and/or velocity 702 being measured by the ear-wearable device. It will be appreciated that in many scenarios, the rotational acceleration and/or velocity may not be purely in sagittal (or vertical), transverse (or horizontal), or frontal planes, but rather occurring as a combination of sagittal, transverse, and frontal at any specific angle as may occur.

In various embodiments herein, ear-wearable devices and/or systems including the same can be used to accurately calculate/estimate linear acceleration and/or velocity at a center of gravity of the head of a wearer of the ear-wearable device.

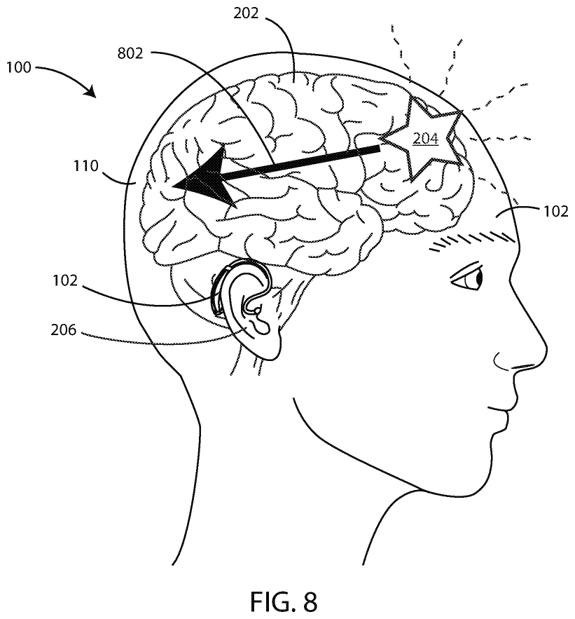

Referring now to FIG. 8, a schematic view of an ear-wearable device 102 being worn is shown in accordance with various embodiments herein. In particular, FIG. 8 shows the head 110, brain 202, and an ear 206 of the device wearer 100. FIG. 8 depicts an injury 204 that has occurred to the brain 202. FIG. 8 shows a vector representing linear acceleration 802 at a center of gravity of the head. In various embodiments, the ear-wearable device 102 in configured to process signals from the motion sensor to estimate linear acceleration 802 at a center of gravity of the head 110 of a wearer of the ear-wearable device 102.

In some embodiments, it can be assumed that the center of gravity of the head 110 is at a midpoint between a first ear-wearable device being worn on one ear and a second ear-wearable device being worn on the other ear. Based on this assumption, the linear acceleration at the center of gravity of the head can be estimated based on averaging of the measured linear acceleration at each of first ear-wearable device and the second ear-wearable device.

In some embodiments, an accessory device can pick up motion that is associated with another part of an individual's body other than the head. This can be useful to analyze as it can provide a more complete picture of the possible damage associated with the impact event. As a specific example, in various embodiments, the ear-wearable device can be configured to estimate a degree of possible damage to a neck of the ear-wearable device wearer based on a comparison of signals from a motion sensor and signals from an accessory device.

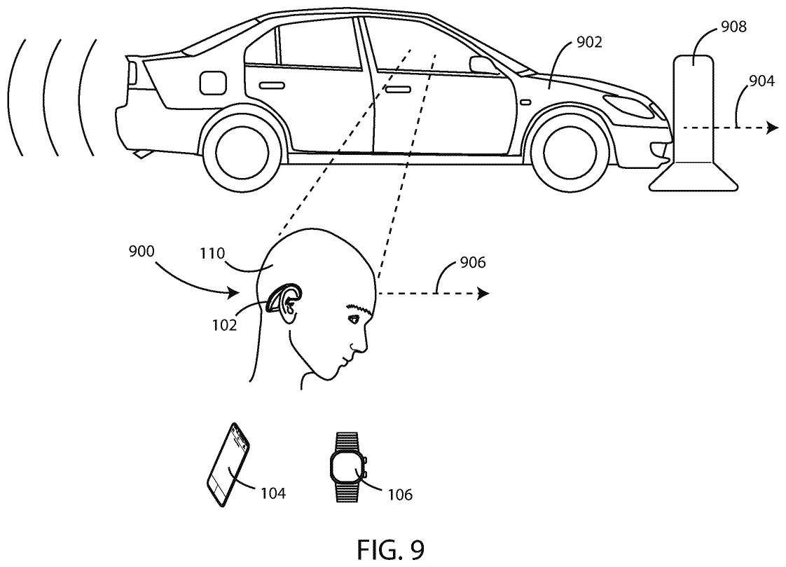

Referring now to FIG. 9, a schematic view of an ear-wearable device 102 being worn by a driver 900 of a vehicle 902 is shown in accordance with various embodiments herein. As the vehicle 902 is traveling, the head 110 includes head speed 906 that is roughly the same direction and speed as vehicle speed 904. However, when the vehicle 902 hits a stationary barrier 908, the vehicle speed 904 is abruptly reduced causing the head 110 to pitch forward. However, the body of the driver 900 may be restrained by a seat belt or similar safety device. This causes the forward speed of the body to be abruptly reduced faster than the head speed 906. FIG. 9 shows an accessory device 104 and a secondary wearable device 106. One or both of the accessory device 104 and the secondary wearable device 106 can be equipped with motion sensors and data from the same can be transferred to the ear-wearable device and/or motion sensor data from the ear-wearable device can be transferred to the accessory device 104 and the secondary wearable device 106. A comparison of the motion sensor can be used to determine the forces differentially experienced by the head versus the body. The ear-wearable device can be configured to estimate a degree of possible damage to a neck of the ear-wearable device wearer where the greater the differential, the greater the likelihood of damage to the neck of the ear-wearable device wearer.

After impact with the stationary barrier 908, the head 110 of the driver 900 may travel forward in the vehicle 902 cab until impacting an air bag or a solid structure. Impact with the air bag or a solid structure may be sufficient to result in head trauma and thus being able to accurately track the same can be of significant diagnostic value. In some embodiments, the ear-wearable device 102 can be configured to monitor signals from the microphone to detect a possible impact event. For example, impact with stationary barrier 908 may generate substantial noise that can be picked up with a microphone associated with the ear-wearable device 102. This can trigger actions as described elsewhere herein such as one or more of changing a sampling rate of one or more sensors, turning on or activating a sensor on that may otherwise be turned off or inactive, changing the nature and/or quantity of data that is stored, or the like.

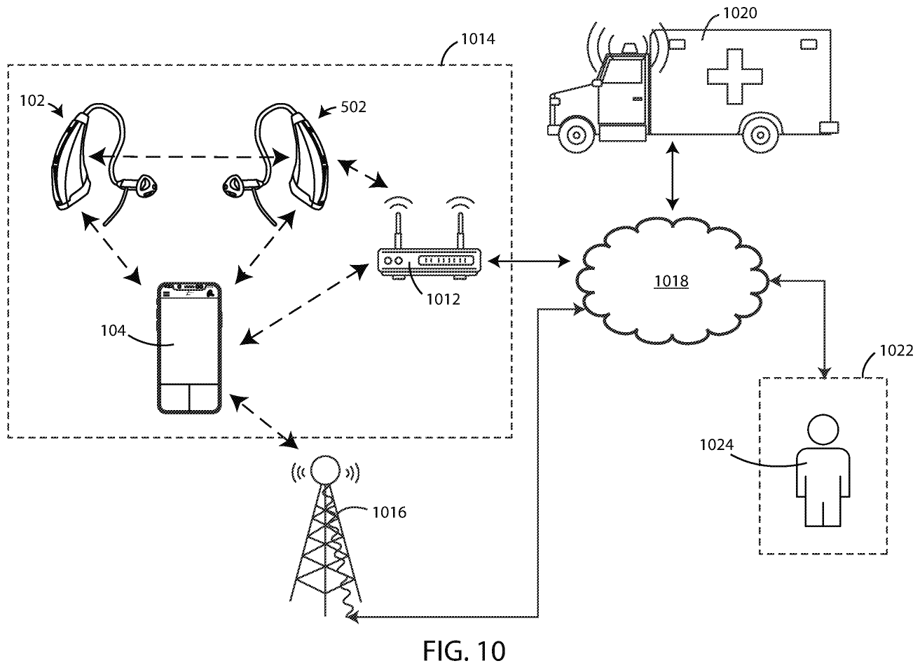

Referring now to FIG. 10, a schematic view is shown of data and/or signal flow as part of a system in accordance with various embodiments herein. In a first location 1014, a device wearer can be wearing a first ear-wearable device 102 and a second ear-wearable device 502. Each of the ear-wearable devices 102, 502 can include sensor packages as described herein including, for example, an IMU. The ear-wearable devices 102, 502 and sensors therein can be disposed on opposing lateral sides of the subject's head. In some embodiments, the ear-wearable devices 102, 502 and sensors therein can be disposed in a fixed position relative to the subject's head. In some embodiments, the ear-wearable devices 102, 502 and sensors therein can be disposed at least partially within opposing ear canals of the subject. The ear-wearable devices 102, 502 and sensors therein can be disposed on or in opposing ears of the subject. The ear-wearable devices 102, 502 and sensors therein can be spaced apart from one another by a distance of at least 3, 4, 5, 6, 8, 10, 12, 14, or 16 centimeters and less than 40, 30, 28, 26, 24, 22, 20 or 18 centimeters, or by a distance falling within a range between any of the foregoing.

In various embodiments, data and/or signals can be exchanged directly between the first ear-wearable device 102 and the second ear-wearable device 502. An accessory device 104 (which could be an external visual display device with a video display screen, such as a smart phone amongst other things) can also be disposed within the first location 1014. The accessory device 104 can exchange data and/or signals with one or both of the first ear-wearable device 102 and the second ear-wearable device 502 and/or with an accessory to the ear-wearable devices (e.g., a remote microphone, a remote control, a phone streamer, etc.). The accessory device 104 can also exchange data across a data network to the cloud 1018, such as through a wireless signal connecting with a local gateway device, such as a network router 1012, mesh network, or through a wireless signal connecting with a cell tower 1016 or similar communications tower. In some embodiments, the external visual display device can also connect to a data network to provide communication to the cloud 1018 through a direct wired connection.

In some embodiments, a third party 1024 (such as a care provider, clinician, physician, specialist, loved one, coach, trainer, or the like) can receive information from devices at the first location 1014 remotely at a second location 1022 through a data communication network such as that represented by the cloud 1018. In some embodiments, the first location 1014 and the second location 1022 are a distance of meters or kilometers apart.

The received information at the second location 1022 can include, but is not limited to, information regarding the nature or severity of the incident of head trauma. In some embodiments, received information can be provided to the third party 1024 in real time. In some embodiments, received information can be stored and provided to the third party 1024 at a time point after response times are measured.

In some embodiments, the third party 1024 can send information remotely from the second location 1022 through a data communication network such as that represented by the cloud 1018 to one or more devices at the first location 1014. The sent information can include, but is not limited to, instructions, queries, and the like.

In various embodiments, the ear-wearable device 102 can be configured to generate an audio warning or alert and send the same to the responsible third party 1024 when an occurrence of head 110 movement consistent with possible head 110 trauma can be detected.

In various embodiments, the ear-wearable device 102 can be configured to select an appropriate first target recipient for the alert. In various embodiments, the ear-wearable device 102 can be configured to select an appropriate first target recipient for the alert based on a predicted severity of possible head 110 trauma. For example, in some scenarios, minutes or event seconds can matter when summoning assistance. As such, in some embodiments, a notification of an incident of serious head trauma can be directed first to an emergency responder 1020. After an emergency responder 1020 is summoned, then a notification or alert can be sent to the responsible third party 1024.

In various embodiments, the ear-wearable device 102 can be configured to stream data based on the signals from at least one of the microphone and the motion sensor after detection an occurrence of head 110 movement consistent with possible head 110 trauma. This can allow a responsible third party 1024 and/or an emergency responder 1020 to gather additional information about the nature of the head trauma experienced by the device wearer while they are still remote from the device wearer. In some embodiments, such as where the head trauma occurs as part of an assault or physical abuse, the streamed data can be used to aid in solving the crime and/or identifying responsible parties based on streamed and recorded voices and/or dialogue.

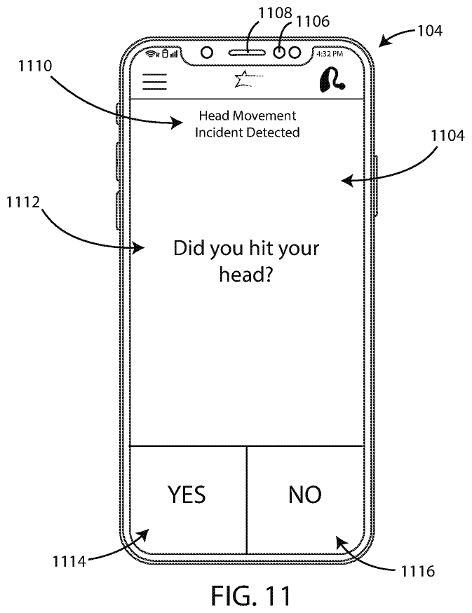

In various embodiments, the ear-wearable device 102 can be configured to query the device wearer 100. Referring now to FIG. 11, a schematic view of an accessory device 104 is shown in accordance with various embodiments herein. The accessory device 104 can include display screen 1104, camera 1106, speaker 1108, notification 1110, query 1112, first user input button 1114, and second user input button 1116. In this example, the ear-wearable device 102 can be configured to query the device wearer 100 if an incident of possible head trauma detected. For example, in some cases the query could be as simple as “Did you hit your head?” as shown in FIG. 11. The individual can then respond by interfacing with one of the user input buttons or simply speaking their answer. In some embodiments, a response indicating that the individual is not feeling well, is nauseous, is feeling numb, is feeling dizzy, is experiencing fatigue, is experiencing problems with their vision, is experiencing changes in hearing or perception of tinnitus, is feeling pain, or is experiencing another symptom of a head injury can be taken as part of an indication or pattern that the individual may have suffered a head injury.

It will be appreciated that queries can take on many different forms. In some embodiments, the query can be visual, aural, tactile or the like. In some embodiments, the query can request device wearer feedback or input (such as could be provided through a button press, an oral response, a movement, etc.). In some embodiments, the query can take the form of a question regarding how the device wearer 100 is feeling or what they are experiencing. In some embodiments, the query can relate to whether they are experiencing dizziness or problems with their vision. In some embodiments, the query can take the form of a question which requires a degree of cognition in order to answer, such as a math question, a verbal question, a question about their personal information (such as one for which the answer is already known by the system), or the like. In some cases, the query can target a response which tests a specific function/area of the brain (e.g., a specific language ability like differentiating phonological or semantic differences between test stimuli). In some cases, there can be a single query. In some cases, there can be multiple queries.

In some embodiments, the query can be delayed. For example, in some embodiments, the query can be queued to be administered to the device wearer at a time point such as 1, 2, 3, 4, 5, 10, 15, 20, 30, 45, or 60 minutes or longer after the possible head injury is detected.

In some embodiments, the ear-wearable device 102 can be configured to evaluate a nature or quality of a response from the device wearer 100 in response to the query. For example, in the context of a question, the system can evaluate whether the answer to the question suggests they are feeling ill or experiencing a symptom of a head injury. As another example, the system can evaluate whether the answer to a question is correct or not. As another example, the system can evaluate the amount of time taken for the device wearer to answer a question. Of course, in some cases a device wearer may simply not respond to a query. In some embodiments, the system can interpret the lack of a response as being indicative of one or more of an occurrence, prodrome or sequelae of a head injury. However, in other embodiments, the system can be configured to not interpret the lack of a response that way. In some embodiments, the system can be configured to allow the user to cease or skip further testing.

As referenced above, detection of circumstances that may precede an impact event, even if the timing of the detection is not sufficient to warn the device wearer, can still be useful as it can allow the ear-wearable device to take steps to enhance detection and/or assessment of possible head trauma. For example, the sampling rate of various sensors can be increased to provide a richer data set to more accurately detect peak acceleration and/or peak velocity. As another example, additional sensors can be turned on to produce a richer data set to more accurately detect possible head trauma. In some cases, an impact event may be preceded by unique movement of the device wearer. For example, a fall of the device wearer may precede a head impact event. As such, in various embodiments, the ear-wearable device can be configured to monitor signals from a motion sensor in order to detect circumstances preceding a possible impact event.

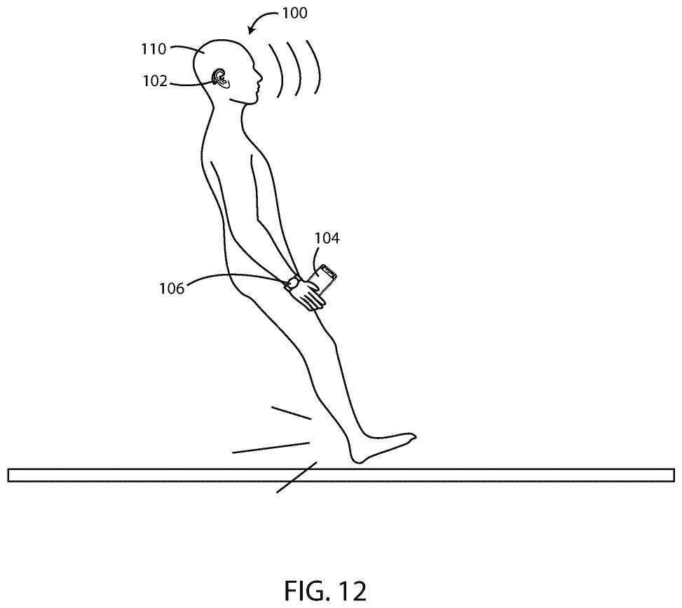

Referring now to FIG. 12, a schematic view of an ear-wearable device wearer 100 falling is shown in accordance with various embodiments herein. FIG. 12 shows a device wearer 100 that is wearing an ear-wearable device 102 on or about their head 110, such as on or in their ear. FIG. 12 also shows an accessory device 104 and a secondary wearable device 106. In this view, the device wearer 100 may have slipped and is currently falling backward. This falling motion can be detected by the ear-wearable device wearer 100 and/or a system including the same. The detection of the falling motion can initiate various actions. By way of example, the sampling rate of various sensors can be increased to provide a richer data set to more accurately detect peak acceleration and/or peak velocity associated with a possible head impact occurring as a result of the fall. By way of example, in some embodiments, a sampling rate of an accelerometer can be increased to at least about 100 Hz, 250 Hz, 500 Hz, 1 kHz, 2 kHz, 3 kHz, 5 kHz, 7 kHz, 10 kHz, 15 kHz, 20 kHz, 30 kHz or higher, or a sampling rate falling within a range between any of the foregoing. In some embodiments, one or more additional sensors can be turned on. By way of example, a gyroscope (which may consume more energy than an accelerometer) can be turned on and data from the same can be recorded. A gyroscope can be used, for example, to measure rotational (or angular) acceleration and/or velocity. In some embodiments, one or more physiological sensors can be turned on and data from the same can be recorded.

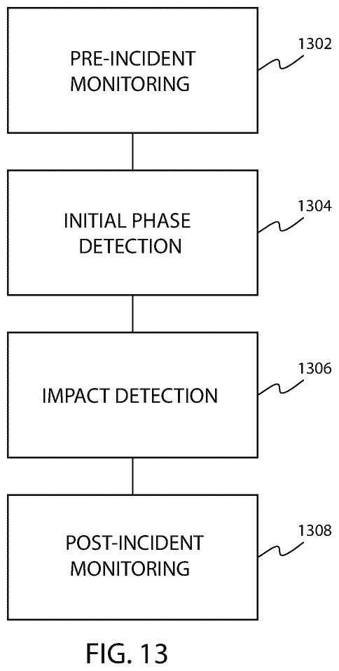

Referring now to FIG. 13, a flow chart of operations of a method is shown in accordance with various embodiments herein. FIG. 13 shows operations associated with pre-incident monitoring 1302. Pre-incident monitoring 1302 can include normal operation of the ear-wearable device. In many cases the ear-wearable device can be powered by a battery. As such, conserving battery power can be a goal when the ear-wearable device and/or system is operating at the stage of pre-incident monitoring 1302 and this can include lower sampling rates for various sensors and/or putting sensors that consume significant electrical power in an “off” or standby state.

FIG. 13 also shows operations associated with an initial phase 1304 of an incident of head trauma. The initial phase 1304 can start with the detection of conditions that make it likely that head trauma is imminent. This can include detection of a fall event such as described with reference to FIG. 12. This can also include detection of sounds associated with an impact, such as a vehicular accident such as described with reference to FIG. 9. The initial phase 1304 can include increasing the sampling rate of one or more sensors. The initial phase 1304 can also include changing storage operations such that more data and/or higher resolution data is stored.

FIG. 13 also illustrates operations associated with an impact event 1306. Operations associated with an impact event 1306 can specifically include identifying a peak acceleration and/or peak velocity value. The peak acceleration and/or peak velocity value can be used to estimate a severity of possible head trauma. Peak acceleration and/or peak velocity can include peak rotational acceleration or peak rotational velocity respectively. Peak acceleration and/or peak velocity can also include peak linear acceleration.

FIG. 13 shows a post-incident monitoring 1308. For example, in various embodiments, the ear-wearable device 102 can be configured monitor signals from the motion sensor to detect at least one of dizziness or a fall after detecting an occurrence of head 110 movement consistent with possible head 110 trauma. In various embodiments, post-incident monitoring 1308 data includes at least one of microphone signals and motion sensor signals. By way of example, the microphone signals can be used to identify slurred speech of the device wearer or other signs of head injury. The motion sensor signals can be used to identify physical manifestations of a head injury such as lack of coordination in physical movements, tremors, shaking, and the like.

The ear-wearable device, and/or system including the same, can predict severity based on factors including one or more of peak detected acceleration, peak detected velocity, post-incident monitoring data, pre-incident monitoring data, and initial phase detection.

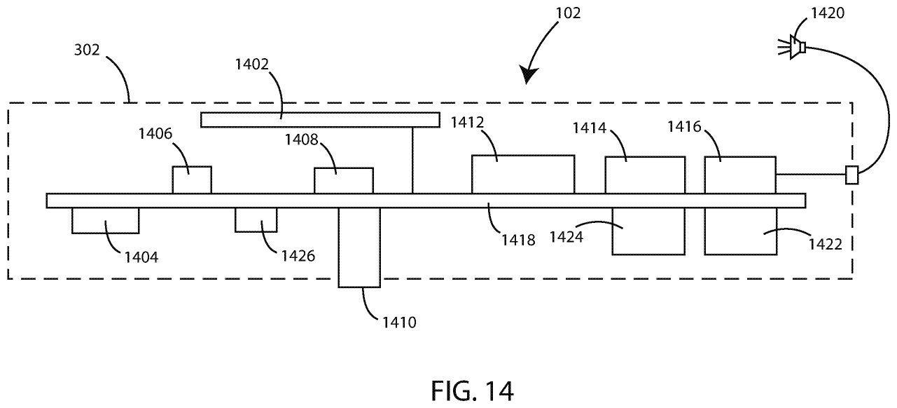

Referring now to FIG. 14, a schematic block diagram of components of an ear-wearable device is shown in accordance with various embodiments herein. The block diagram of FIG. 14 represents a generic ear-wearable device for purposes of illustration. The ear-wearable device 102 shown in FIG. 14 includes several components electrically connected to a flexible mother circuit 1418 (e.g., flexible mother board) which is disposed within housing 302. A power supply circuit 1404 can include a battery and can be electrically connected to the flexible mother circuit 1418 and provides power to the various components of the ear-wearable device 102. One or more microphones 1406 are electrically connected to the flexible mother circuit 1418, which provides electrical communication between the microphones 1406 and a digital signal processor (DSP) 1412. Among other components, the DSP 1412 incorporates or is coupled to audio signal processing circuitry configured to implement various functions described herein. A sensor package 1414 can be coupled to the DSP 1412 via the flexible mother circuit 1418. The sensor package 1414 can include one or more different specific types of sensors such as those described in greater detail below. One or more user switches 1410 (e.g., on/off, volume, mic directional settings) are electrically coupled to the DSP 1412 via the flexible mother circuit 1418.

An audio output device 1416 is electrically connected to the DSP 1412 via the flexible mother circuit 1418. In some embodiments, the audio output device 1416 comprises an electroacoustic transducer or speaker (coupled to an amplifier). In other embodiments, the audio output device 1416 comprises an amplifier coupled to an external receiver 1420 adapted for positioning within an ear of a wearer. The external receiver 1420 can include an electroacoustic transducer, speaker, or loudspeaker.

The ear-wearable device 102 may incorporate a communication device 1408 coupled to the flexible mother circuit 1418 and to an antenna 1402 directly or indirectly via the flexible mother circuit 1418. The communication device 1408 can be a BLUETOOTH® transceiver, such as a BLE (BLUETOOTH® low energy) transceiver or other transceiver(s) (e.g., an IEEE 802.11 compliant device). The communication device 1408 can be configured to communicate with one or more external devices, such as those discussed previously, in accordance with various embodiments. In various embodiments, the communication device 1408 can be configured to communicate with an external visual display device such as a smart phone, a video display screen, a tablet, a computer, or the like.

In some embodiments, ear-wearable devices 102 of the present disclosure can incorporate an antenna arrangement coupled to a high-frequency radio, such as a 2.4 GHz radio. The radio can conform to an IEEE 802.11 (e.g., WIFI®) or BLUETOOTH® (e.g., BLE, BLUETOOTH® 4.2 or 5.0) specification, for example. It is understood that ear-wearable devices of the present disclosure can employ other radios, such as a 900 MHz radio or radios operating at other frequencies or frequency bands. Ear-wearable device of the present disclosure can also include hardware, such as one or more antennas, for NFMI or NFC wireless communications. Ear-wearable devices of the present disclosure can be configured to receive streaming audio (e.g., digital audio data or files) from an electronic or digital source.

Ear-wearable devices 102 of the present disclosure can be configured to receive streaming audio (e.g., digital audio data or files) from an electronic or digital source. Representative electronic/digital sources (also referred to herein as accessory devices) include an assistive listening system, a TV streamer, a radio, a smartphone, a cell phone/entertainment device (CPED) or other electronic device that serves as a source of digital audio data or files. Systems herein can also include these types of accessory devices as well as other types of devices.

In various embodiments, the ear-wearable device 102 can also include a control circuit 1422 and a memory storage device 1424. The control circuit 1422 can be in electrical communication with other components of the device. In some embodiments, a clock circuit 1426 can be in electrical communication with the control circuit. The control circuit 1422 can execute various operations, such as those described herein. The control circuit 1422 can include various components including, but not limited to, a microprocessor, a microcontroller, an FPGA (field-programmable gate array) processing device, an ASIC (application specific integrated circuit), or the like. The memory storage device 1424 can include both volatile and non-volatile memory. The memory storage device 1424 can include ROM, RAM, flash memory, EEPROM, SSD devices, NAND chips, and the like. The memory storage device 1424 can be used to store data from sensors as described herein and/or processed data generated using data from sensors as described herein.

It will be appreciated that various of the components described in FIG. 14 can be associated with separate devices and/or accessory devices to the ear-wearable device. By way of example, microphones can be associated with separate devices and/or accessory devices. Similarly, audio output devices can be associated with separate devices and/or accessory devices to the ear-wearable device.

Accessory devices herein can include various different components. In some embodiments, the accessory device can be a personal communications device, such as a smart phone. However, the accessory device can also be other things such as a secondary wearable device, a handheld computing device, a dedicated location determining device (such as a handheld GPS unit), or the like.

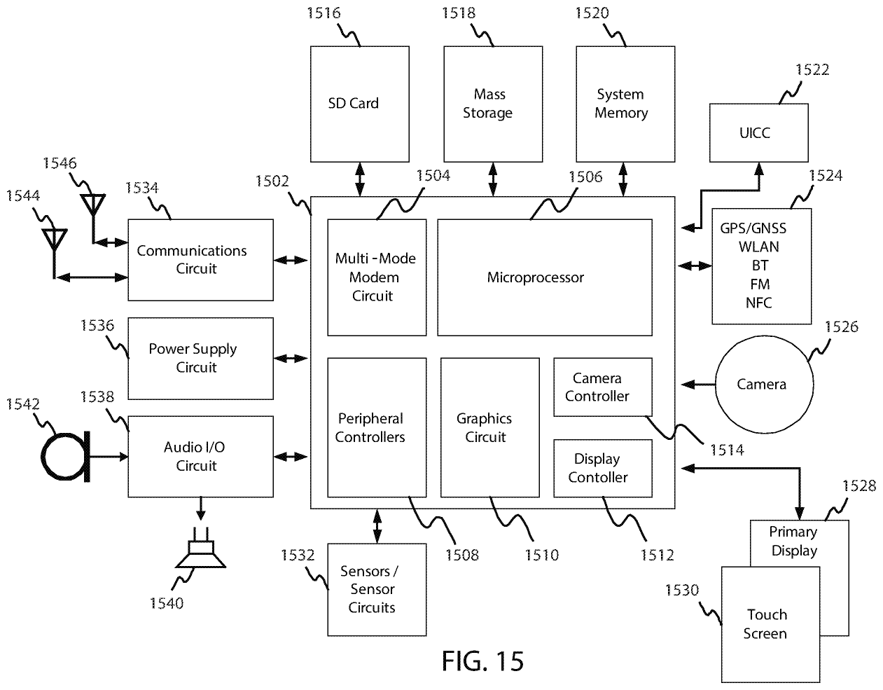

Referring now to FIG. 15, a schematic block diagram is shown of components of an accessory device 104 (which could be a personal communications device or another type of accessory device) in accordance with various embodiments herein. This block diagram is just provided by way of illustration and it will be appreciated that accessory devices can include greater or lesser numbers of components. The accessory device in this example can include a control circuit 1502. The control circuit 1502 can include various components which may or may not be integrated. In various embodiments, the control circuit 1502 can include a microprocessor 1506, which could also be a microcontroller, FPGA, ASIC, or the like. The control circuit 1502 can also include a multi-mode modem circuit 1504 which can provide communications capability via various wired and wireless standards. The control circuit 1502 can include various peripheral controllers 1508. The control circuit 1502 can also include various sensors/sensor circuits 1532. The control circuit 1502 can also include a graphics circuit 1510, a camera controller 1514, and a display controller 1512. In various embodiments, the control circuit 1502 can interface with an SD card 1516, mass storage 1518, and system memory 1520. In various embodiments, the control circuit 1502 can interface with universal integrated circuit card (UICC) 1522. A spatial location determining circuit can be included and can take the form of an integrated circuit 1524 that can include components for receiving signals from GPS, GLONASS, BeiDou, Galileo, SBAS, WLAN, BT, FM, NFC type protocols, 5G picocells, or E911. In various embodiments, the accessory device can include a camera 1526. In various embodiments, the control circuit 1502 can interface with a primary display 1528 that can also include a touch screen 1530. In various embodiments, an audio I/O circuit 1538 can interface with the control circuit 1502 as well as a microphone 1542 and a speaker 1540. In various embodiments, a power supply circuit 1536 can interface with the control circuit 1502 and/or various other circuits herein in order to provide power to the system. In various embodiments, a communications circuit 1534 can be in communication with the control circuit 1502 as well as one or more antennas (1544, 1546).

In some embodiments, image/video data as recorded by an accessory device can be used in combination with other sensor data in order to more fully capture the incident resulting in head trauma. For example, image/video data can be correlated with other sensor data and the combined data set can be used to more fully understand the trauma that may have occurred to the brain of the device wearer. In some embodiments, image/video data can be used in order to identify a surface or object which impacts the head of the wearer causing the potential head trauma. This can provide useful information to a clinician or other third party for both treatment of the head trauma as well as prevention of future head trauma.

Ear-wearable devices herein can take on many different forms. In some embodiments, the ear-wearable device can be in the form of an in-the-ear style custom ear-wearable device. While not intending to be bound by theory, it is believed that certain form factors, such as an in-the-ear style custom ear-wearable device, can have better mechanical coupling to the head which can be advantageous because more solid attachment can provide more accurate data. Additionally, the retention of the ear-wearable device during and after a TBI incident is more likely with an in-the-ear style custom ear-wearable device.

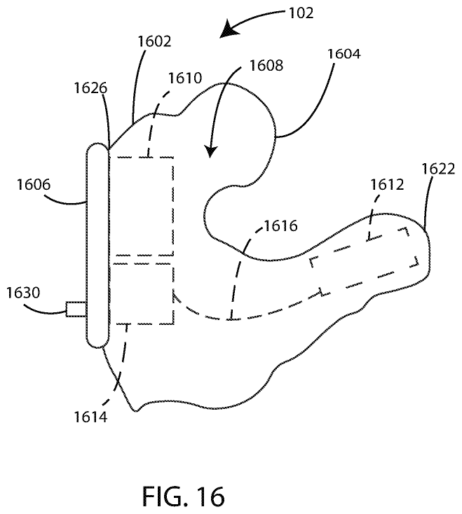

Referring now to FIG. 16, a schematic view of an in-the-ear style custom ear-wearable device 102 is shown in accordance with various embodiments herein. The ear-wearable device 102 can include an ear-wearable device housing 1602 formed by a shell 1604 and a faceplate 1606. The shell 1604 is custom shaped to mate with the user's ear anatomy and defines an internal shell cavity 1608 and a shell aperture at the entrance to the shell cavity 1608. The faceplate 1606 is attached to the shell at the shell aperture to enclose the shell cavity 1608.

The ear-wearable device housing 1602 can define a battery compartment 1610 in which a battery can be disposed to provide power to the device. The ear-wearable device 102 can also include a receiver 1612. The receiver 1612 can include a component that converts electrical impulses into sound, such as an electroacoustic transducer, speaker, or loudspeaker. The housing 1602 can also define a component compartment 1614 that can contain electrical and other components including but not limited to a microphone, a processor, memory, various sensors, one or more communication devices, power management circuitry, and a control circuit. A cable 1616 or connecting wire can include one or more electrical conductors and provide electrical communication between components inside of the component compartment 1614 and components inside of the receiver 1612.

The shell 1604 extends from an ear canal end 1622 to an aperture end 1626. At the aperture end 1626, the shell 1604 defines an aperture that is closed by the faceplate 1606. The faceplate 1606 is sealed to the shell 1604. The faceplate 1606 is shown in FIG. 16 only in a side view but can include many features and structures. A user input device 1630 is shown as part of the faceplate in FIG. 16, and can be a button, lever, switch, dial, or other input device. The faceplate 1606 may also include a battery door, a microphone opening, a pull handle, and other features.

The ear-wearable device 102 shown in FIG. 16 is an in-the-ear style device and thus the shell is designed to be placed within the ear cavity. However, it will be appreciated that many different form factors for ear-wearable devices are contemplated herein. Aspects of ear-wearable devices and functions thereof are described in U.S. Pat. No. 9,848,273; U.S. Publ. Pat. Appl. No. 20180317837; and U.S. Publ. Pat. Appl. No. 20180343527, the content of all of which is herein incorporated by reference in their entirety.

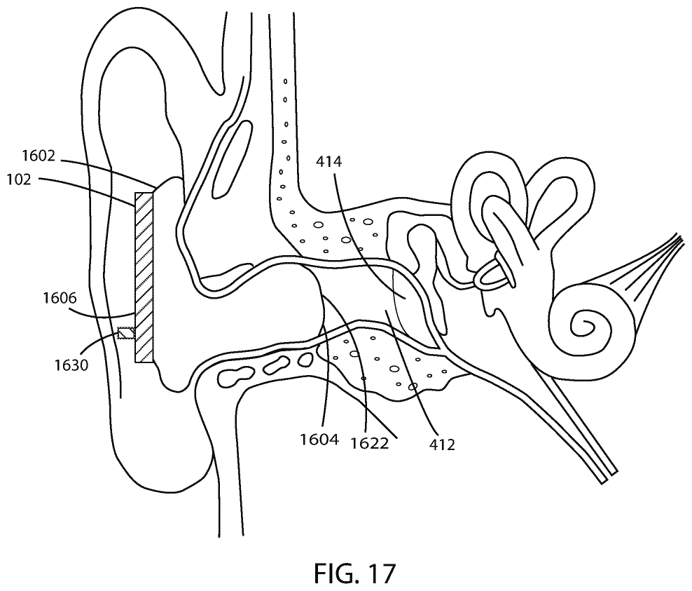

FIG. 17 is a schematic view of an ear-wearable device 102 disposed within the ear of a wearer in accordance with various embodiments herein. The housing 1602 of the ear-wearable device 102 is defined by the shell 1604, which is positioned within the ear canal 412, and the faceplate 1606, which is positioned in the concha. The user input device 1630 on the faceplate 1606 is accessible to be manipulated by the user without having to remove the ear-wearable device from their ear. The ear canal end 1622 of the shell 1604 is positioned close to the user's tympanic membrane. Ideally, the shell 1604 fits properly within the user's ear cavity. A proper fit is usually one in which the ear-wearable device forms an acoustic seal with the user's ear cavity, so that it is contacting the ear cavity around a circumference of the ear-wearable device at some location on the shell 1604 of the ear-wearable device 102. A proper fit is also comfortable to the user, so that the shell 1604 is not putting too much pressure on the walls of the ear canal 412 or features of the concha. The receiver 1612 (FIG. 16) is positioned within the shell 1604 at the ear canal end 1622 of the shell 1604 to minimize the distance between the receiver 1612 and the tympanic membrane 414 without physically contacting the tympanic membrane 414.

### Methods

Many different methods are contemplated herein, including, but not limited to, methods of identifying, tracking, or monitoring head trauma, methods operating an ear-wearable device, methods of using an ear-wearable device, and the like. Aspects of system/device operation described elsewhere herein can be performed as operations of one or more methods in accordance with various embodiments herein.

In an embodiment, a method of tracking head trauma with an ear-wearable device is included. The method can include evaluating signals from a motion sensor that is part of the ear-wearable device, calculating at least one of peak rotational acceleration, peak linear acceleration, and peak rotational velocity, and comparing such peak values against one or more threshold values for head trauma. In some embodiments, the method can further include recording calculated peak values. In some embodiments of the method, recording calculated peak values further comprises calculating a cumulative impact index based on the recorded peak acceleration and/or peak velocity values.

In some embodiments, other pieces of data other than peak acceleration and/or peak velocity can also be recorded. For example, other data can include head orientation, neck orientation, head rotation, head movements in all directions, and the like, and all of the preceding over time including acceleration over time and velocity over time.

In some embodiments, the recorded data can be sufficient for and can be used to create a 3-D simulation of the incident of head trauma. For example, the recorded data can be used along with finite element analysis techniques in order to recreate the events leading up to, during, and after the head injury and/or recreate the brain deformation and potential brain damage resulting from an impact or other incident of head trauma. Recreating the events can lead to better understanding for a clinician of the potential effects of the head injury on the brain. This can lead to more accurate diagnosis as well as provide for statistical data to correlate the conditions of the incident (including the type and degree of observed acceleration and/or velocity) to resulting brain injury. In addition, the creation of such simulations or models is very valuable in studying brain injury since there is no high acceleration head impact experimental data on human subjects. Various anatomical structures of the brain can be modeled including, but not limited to, white matter, gray matter, caudate, putamen, thalamus, ventricles, cerebrospinal fluid (CSF), and the subarachnoidal space (SAS), and the forces, accelerations, and/or velocities of the same can be visualized with such a 3-D simulation. In some embodiments, a material point method (MPM) can be utilized to create a 3-D simulation of the incident instead of, or in addition to, a finite element approach. In some embodiments, the recorded data can be used to populate a publicly available computational model of the human head.

In an embodiment, the method can further include changing a sampling rate of the motion sensor if movement is detected consistent with an initial phase of an impact event. In an embodiment, the method can further include increasing a sampling rate of the motion sensor if movement is detected consistent with an initial phase of an impact event.

In an embodiment, the method can further include monitoring signals from the motion sensor to detect at least one of dizziness or a fall after detecting an occurrence of peak acceleration and/or peak velocity consistent with possible head trauma.

In an embodiment, the method can further include generating an audio warning when an occurrence of peak acceleration and/or peak velocity consistent with possible head trauma is detected. In an embodiment, the method can further include generating an audio warning and sending the same to a responsible third party when an occurrence of peak acceleration and/or peak velocity consistent with possible head trauma is detected.

In an embodiment, the method can further include generating a query for a wearer of the ear-wearable device when an occurrence of peak acceleration and/or peak velocity consistent with possible head trauma is detected. In an embodiment, the method can further include generating a delayed query for a wearer of the ear-wearable device when an occurrence of peak acceleration and/or peak velocity consistent with possible head trauma is detected.

In an embodiment, the method can further include estimating linear acceleration at a center of gravity of the head of a wearer of the ear-wearable device.

In an embodiment, the method can further include evaluating signals from a microphone that is part of the ear-wearable device to detect a possible impact event.

In an embodiment, the method can further include receiving signals regarding detected motion from an accessory device. In an embodiment of the method, receiving signals regarding detected motion from an accessory device further comprises estimating a degree of possible damage to a neck of the ear-wearable device wearer based on a comparison of signals from the motion sensor and signals from the accessory device.

In an embodiment, the method can further include generating an alert if an occurrence of possible head trauma is detected that crosses a threshold value.

In an embodiment, the method can further include selecting an appropriate first target recipient for the alert. In an embodiment, the method can further include selecting an appropriate first target recipient for the alert based on a predicted severity of possible head trauma.

In an embodiment, the method can further include predicting severity based on factors including one or more of peak detected acceleration, peak detected velocity, post-incident monitoring data, pre-incident monitoring data, and initial phase detection.

In an embodiment of the method, post-incident monitoring data includes at least one of microphone signals and motion sensor signals.

In an embodiment, the method can further include evaluating sensor data from at least one sensor selected from the group consisting of a blood pressure sensor and a heart rate sensor.

In an embodiment, the method can further include streaming data based on signals from at least one of the motion sensor and a microphone after detection an occurrence of head movement consistent with possible head trauma.

### False-Positive Detection/Mitigation

If an ear-wearable device is dropped, the resulting accelerations may be confused with a head impact creating head trauma. As such, dropping an ear-wearable device can create a false positive. However, in various embodiments herein, the device and/or system can be configured to mitigate false positive events. For example, in various embodiments herein, the device and/or system can evaluate signals associated with one or more sensors to detect whether such signals are indicative of the ear-wearable devices simply being dropped (e.g., dropped to the ground or another surface when not being worn on, in or about the ears by the device wearer, or after falling off of or out of the ears).

For example, in various embodiments, signals from a temperature sensor, a physiological sensor (including, but not limited to a heart rate sensor), a capacitive sensor, an impedance sensor, an electrical potential sensor, or another type of sensor exhibits a detectable change when the ear-wearable device are no longer being worn by the device wearer. For example, a temperature sensor would indicate a drop in temperature. An electrical potential sensor or a capacitive sensor would exhibit values that indicate the sensor is no longer in contact with the skin. As such, in various embodiments herein, the ear-wearable device is configured to monitor signals from the motion sensor to detect an occurrence of head movement consistent with possible head trauma and also monitor signals from another sensor in order to detect whether the ear-wearable devices are no longer being worn. If the signals from the other sensor(s) are consistent with the ear-wearable device no longer being worn, then the ear-wearable device can be configured to ignore or otherwise not utilize the signals from the motion sensor (or another sensor) that would otherwise be consistent with a head trauma event.

In some embodiments, such as in the case of a system including two ear-wearable devices, the motion (including acceleration and/or velocity) from two individual devices can be compared with one another or correlated. If only one device reports motion consistent with possible head trauma, then it can be interpreted as a false positive event.

### Head Trauma Detection

Various embodiments herein include a head trauma detection. Further details about the head trauma detection are provided as follows. However, it will be appreciated that this is merely provided by way of example and that further variations are contemplated herein.

In various embodiments, signals from the motion sensor that cross a threshold value of peak acceleration and/or peak velocity are treated as being consistent with possible head trauma. Specifically, in some embodiments signals from the motion sensor that cross a threshold value of peak rotational acceleration and/or velocity are deemed to be consistent with possible head trauma. In some embodiments signals from the motion sensor that cross a threshold value of peak linear acceleration are deemed to be consistent with possible head trauma. The thresholds for rotational acceleration/velocity versus linear acceleration can be different beyond just being measured in different units. For example, it is believed that rotational acceleration and/or velocity can be more harmful in terms of head trauma than linear acceleration. As such, in various embodiments, the threshold for peak rotational acceleration and/or velocity can be, relatively speaking, lower than the threshold for peak linear acceleration.

In some embodiments, a threshold value for rotational acceleration in rad/s² that is considered to be indicative for head trauma can be about 1,000, 2,000, 3,000, 4,000, 5,000, 6,000, 7,000, or 8,000 rad/s², or an amount falling within a range between any of the foregoing.

In some embodiments, a threshold value for rotational velocity (or angular velocity) in rad/s that is considered to be indicative for head trauma can be about 10, 15, 20, 25, 30, 35, 40, 45, or 50 rad/s, or an amount falling within a range between any of the foregoing.

In some embodiments, a threshold value for linear acceleration in units of g that is considered to be indicative for head trauma can be about 20 g, 30 g, 40 g, 50 g, 60 g, 70 g, 80 g, 90 g, or 100 g or an amount falling within a range between any of the foregoing.

In various embodiments, the ear-wearable device is configured to record detected occurrences of head movement consistent with possible head trauma. In some embodiments, the data recorded can include sensor data (or extracts thereof) covering a time period from moments before the impact, during the impact event, and/or after the impact has occurred.

In various embodiments, the ear-wearable device is configured to calculate a cumulative impact index based on the recorded detected occurrences of head movement consistent with possible head trauma. In various embodiments, the index can take into account intensity. For example, values of individual events in the index can be weighted such that impacts with higher peak measured accelerations and/or velocities are weighted more heavily (and therefore increase the index value more) than those impacts with lower peak measured accelerations and/or velocities. In some embodiments, the index can take into account the duration of the event. For example, a head impact with peak rotational acceleration of 10,000 rad/s 2 and a duration of 10 ms can be far more injurious than a head impact with the same peak acceleration, but only a 5 ms duration. As such, the cumulative impact index can weight events of a longer duration more heavily than otherwise similar events that have a shorter duration. In some embodiments, the index can be temporally weighted. For example, values of individual events in the index can be weighted such that additional impacts occurring close in time (e.g., within minutes, hours, or days) are weighted more heavily (and therefore increase the index value more) than those impact occurring at a much later time all things being equal.

In some embodiments, the ear-wearable device can issue alerts or warnings based on the current value of the cumulative impact index. For example, if the device wearer is playing a sport and the current value of the cumulative impact index rises above a threshold value, then an alert or warning can be sent to a responsible athletic trainer or a coach to cause them to take appropriate action to prevent any further head trauma to the device wearer. In some scenarios, an alert or warning can be sent to a referee or responsible official in order to have them consider disqualifying the individual from further play to prevent further injury and/or to consider penalizing an opposing player responsible for the injury. In another example, if the device wearer is a patient in a care facility, if the current value of the cumulative impact index rises above a threshold value, then an alert or warning can be sent to a responsible third party and/or a loved one to cause them to investigate and/or take appropriate action to prevent any further head trauma to the device wearer.

### Fall Detection

As described above, in various embodiments detection of a possible fall can trigger changes to device operation in order to more accurately capture an incident of head trauma. By tracking motion using one or more motion sensors (and in some cases other types of sensors also) and evaluating data from the same, patterns or signatures indicative of a fall can be detected. In some embodiments, patterns or signatures indicative of a fall can include a detected rapid downward movement of a subject's head and/or other body parts (e.g., sudden height change), downward velocity exceeding a threshold value followed by a sudden stop. In some embodiments, patterns or signatures of a fall can include a detected rapid rotation of a subject's head, such as from an upright position to a non-upright position. In various embodiments, patterns or signatures indicative of a fall can include multiple factors including, for example, a rapid downward movement, downward velocity exceeding a threshold value followed by a sudden stop, or a downward rotation of a subject's head and/or other body parts along with other aspects including one or more of the subject's head remaining at a non-upright angle for at least a threshold amount of time, the subject's body in a prone, supine or lying on side position for at least a threshold amount of time, sound indicating an impact, sound indicating a scream, and the like. In some embodiments, the signal strength of wireless communications between various devices may be used to determine the position of an individual, relative to various reflective or absorptive surfaces, at various phases of a fall event, such as the ground.

In some cases, sensor signals can be monitored for a fall and can specifically include classifying pre-fall motion activity, detecting the onset of a falling phase, detecting impacts, and evaluating post-impact activity. To do so, the hearing assistance device can calculate various feature values from motion data, such as vertical acceleration, estimated velocity, acceleration duration, estimated falling distance, posture changes, and impact magnitudes.

Monitoring for a possible fall can include tracking the total acceleration signal (SV_tot) peaks and comparing them against a threshold value, such as see if they are greater than a threshold. In some embodiments, falling detection can include tracking based on smoothed vertical acceleration, estimating vertical velocity, evaluating against thresholds for duration, minimum SV_tot, and vertical velocity, and monitoring the posture change.

The signal of an IMU or accelerometer can be considered as \vec{s}=\vec{a}−\vec{g}, where \vec{s} is the signal, \vec{a} is the acceleration, and −\vec{g} is the bias. The direction of g (gravity) is in the negative z direction, therefore, the bias is in the positive z direction. By determining the directionality of the bias, the direction of gravity can be derived. By knowing the direction of gravity relative to a device, the posture of the device wearer can be derived (e.g., standing, lying face up, lying face down, etc.). In addition, in various embodiments herein, the direction of gravity can be determined and compared between hearing assistance devices. If both devices are being worn, then the direction of gravity should be within a given amount of each other (such as within 10, 5 or 3 degrees). If the direction of gravity is not comparable between the two devices, then this can be taken as an indication that one or both of the devices is no longer being worn by the device wearer. In such a case, data indicating a possible fall can be ignored or otherwise not acted upon by the system, particularly where only one device indicates a possible fall but its indicated direction of gravity has changed with respect to the other device.

In some embodiments, devices (hearing assistance or accessory) and/or systems herein are configured to evaluate data from one or more sensors to detect a possible fall of a subject in physical contact with the hearing assistance device by evaluating at least one of timing of steps and fall detection phases (including, but not limited to a pre-fall phase, a falling phase, an impact phase, and a resting phase), degree of acceleration changes, direction of acceleration changes, peak acceleration changes, activity classification, and posture changes.

In some cases, multiple algorithms for fall detection can be used, with one or more being more highly sensitive and one or more producing fewer false positives.

In some embodiments herein, patterns or signatures of a fall for a particular subject can be enhanced over time through machine learning analysis. For example, the subject (or a third party) can provide input as to the occurrence of falls and/or the occurrence of false-positive events. These occurrences of falls and/or false positives can be paired with data representing data gathered at the time of these occurrences. Then, an approach such as a supervised machine learning algorithm can be applied in order to derive a pattern or signature consistent with a fall and/or a false positive. In this way, the pattern or signature can be updated over time to be more accurate both for a specific subject as well as for a population of subjections. In some embodiments, fall detection sensitivity thresholds may be automatically or dynamically adjusted, for the subject, to capture a greater number of falls as the machine learning techniques improve the system's ability to reject false-positive detections over time. In some embodiments, user input responses regarding whether or not a fall has occurred and/or whether or not the subject sustained an injury as a result of the fall as described previously can be stored with fall data in the cloud and can be used as inputs into machine learning based fall detection algorithm improvement. These data may also be used to calculate statistics relative to the subject's risk for future falls.

In some embodiments, an assessed fall risk can be used as a factor in determining whether a fall is occurring. For example, a fall risk can be calculated according to various techniques, including, but not limited to techniques described in U.S. Publ. Pat. Appl. Nos. 2018/0228405; 2018/0233018; and 2018/0228404, the content of which is herein incorporated by reference. The assessed fall risk can then be applied such that the system is more likely to indicate that a fall has occurred if the assessed fall risk was relatively high immediately before the occurrence in question. In some embodiments, the assessed fall risk can be applied transitorily such that the system is only more likely to indicate that a fall has occurred for a period of seconds or minutes. In other embodiments, the assessed fall risk can be applied over a longer period of time.

In some embodiments, device settings can include a fall detection sensitivity setting such that the subject or a third party can change the device or system settings such that the fall detection criteria becomes more or less sensitive. In some cases, sensitivity control can relate to implementing/not implementing some of the aspects that relate to reducing false positives. In other words, sensitivity control may not be just related to thresholds for sensitivity, but also related to thresholds for specificity.

Further details of fall detection are described in U.S. Publ. Pat. Appl. Nos. 2020/0236479, 2018/0228404, and 2018/0233018, the content of which is herein incorporated by reference.

### Sensors

Ear-wearable devices herein can include one or more sensor packages (including one or more discrete or integrated sensors) to provide data. The sensor package can comprise one or a multiplicity of sensors. In some embodiments, the sensor packages can include one or more motion sensors (or movement sensors) amongst other types of sensors. Motion sensors herein can include inertial measurement units (IMU), accelerometers, gyroscopes, barometers, altimeters, and the like. The IMU can be of a type disclosed in commonly owned U.S. Pat. No. 9,848,273, which is incorporated herein by reference. In some embodiments, electromagnetic communication radios or electromagnetic field sensors (e.g., telecoil, NFMI, TMR, GMR, etc.) sensors may be used to detect motion or changes in position. In various embodiments, the sensor package can include a magnetometer. In some embodiments, biometric sensors may be used to detect body motions or physical activity. Motions sensors can be used to track movement of a patient in accordance with various embodiments herein.

In some embodiments, the motion sensors can be disposed in a fixed position with respect to the head of a patient, such as worn on or near the head or ears. In some embodiments, the operatively connected motion sensors can be worn on or near another part of the body such as on a wrist, arm, or leg of the patient.

According to various embodiments, the sensor package can include one or more of an IMU, and accelerometer (3, 6, or 9 axis), a gyroscope, a barometer, an altimeter, a magnetometer, a magnetic sensor, an eye movement sensor, a pressure sensor, an acoustic sensor, a telecoil, a heart rate sensor, a global positioning system (GPS), a microphone, an acoustic sensor, a wireless radio antenna, an air quality sensor, an optical sensor, a light sensor, an image sensor, a temperature sensor, a physiological sensor such as a blood pressure sensor, an oxygen saturation sensor, a blood glucose sensor (optical or otherwise), a galvanic skin response sensor, a cortisol level sensor (optical or otherwise), an electrocardiogram (ECG) sensor, electroencephalography (EEG) sensor which can be a neurological sensor, eye movement sensor (e.g., electrooculogram (EOG) sensor), myographic potential electrode sensor (EMG), a heart rate monitor, a pulse oximeter or oxygen saturation sensor (SpO2), blood perfusion sensor, hydrometer, sweat sensor, cerumen sensor, pupillometry sensor, hematocrit sensor, or the like.

In some embodiments, the sensor package can be part of an ear-wearable device. However, in some embodiments, the sensor packages can include one or more additional sensors that are external to an ear-wearable device. For example, various of the sensors described above can be part of a wrist-worn or ankle-worn sensor package, or a sensor package supported by a chest strap. In some embodiments, sensors herein can be disposable sensors that are adhered to the device wearer (“adhesive sensors”) and that provide data to the ear-wearable device or another component of the system.

Data produced by the sensor(s) of the sensor package can be operated on by a processor of the device or system.

As used herein the term “inertial measurement unit” or “IMU” shall refer to an electronic device that can generate signals related to a body's specific force and/or angular rate. IMUs herein can include one or more accelerometers (3, 6, or 9 axis) to detect linear acceleration and a gyroscope to detect rotational acceleration and/or velocity. In some embodiments, an IMU can also include a magnetometer to detect a magnetic field.

The eye movement sensor may be, for example, an electrooculographic (EOG) sensor, such as an EOG sensor disclosed in commonly owned U.S. Pat. No. 9,167,356, which is incorporated herein by reference. The pressure sensor can be, for example, a MEMS-based pressure sensor, a piezo-resistive pressure sensor, a flexion sensor, a strain sensor, a diaphragm-type sensor and the like.

The temperature sensor can be, for example, a thermistor (thermally sensitive resistor), a resistance temperature detector, a thermocouple, a semiconductor-based sensor, an infrared sensor, or the like.

The blood pressure sensor can be, for example, a pressure sensor. The heart rate sensor can be, for example, an electrical signal sensor, an acoustic sensor, a pressure sensor, an infrared sensor, an optical sensor, or the like.

The oxygen saturation sensor (such as a blood oximetry sensor) can be, for example, an optical sensor, an infrared sensor, a visible light sensor, or the like.

The electrical signal sensor can include two or more electrodes and can include circuitry to sense and record electrical signals including sensed electrical potentials and the magnitude thereof (according to Ohm's law where V=IR) as well as measure impedance from an applied electrical potential.

It will be appreciated that the sensor package can include one or more sensors that are external to the ear-wearable device. In addition to the external sensors discussed hereinabove, the sensor package can comprise a network of body sensors (such as those listed above) that sense movement of a multiplicity of body parts (e.g., arms, legs, torso).

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. An ear-wearable device comprising:
a control circuit;
a microphone, wherein the microphone is in electrical communication with the control circuit;
a motion sensor, wherein the motion sensor is in electrical communication with the control circuit; and
a power supply circuit, wherein the power supply circuit is in electrical communication with the control circuit;
wherein the ear-wearable device is configured to monitor signals from the motion sensor to detect an occurrence of head movement consistent with possible head trauma.

2. The ear-wearable device of any of claims 1 and 3-26, wherein the ear-wearable device is configured to record detected occurrences of head movement consistent with possible head trauma.

3. The ear-wearable device of any of claims 1-2 and 4-26, wherein signals from the motion sensor that cross a threshold value of acceleration and/or velocity are consistent with possible head trauma.

4. The ear-wearable device of any of claims 1-3 and 5-26, wherein the ear-wearable device is configured to calculate a cumulative impact index based on the recorded detected occurrences of head movement consistent with possible head trauma.

5. The ear-wearable device of any of claims 1-4 and 6-26, wherein the ear-wearable device is configured to change a sampling rate of the motion sensor if movement is detected consistent with an initial phase of an impact event.

6. The ear-wearable device of any of claims 1-5 and 7-26, wherein the ear-wearable device is configured to increase a sampling rate of the motion sensor if movement is detected consistent with an initial phase of an impact event.

7. The ear-wearable device of any of claims 1-6 and 8-26, wherein the ear-wearable device is configured monitor signals from the motion sensor to detect at least one of dizziness or a fall after detecting an occurrence of head movement consistent with possible head trauma.

8. The ear-wearable device of any of claims 1-7 and 9-26, wherein the ear-wearable device is configured to generate an audio warning when an occurrence of head movement consistent with possible head trauma is detected.

9. The ear-wearable device of any of claims 1-8 and 10-26, wherein the ear-wearable device is configured to generate an audio warning and send the same to a responsible third party when an occurrence of head movement consistent with possible head trauma is detected.

10. The ear-wearable device of any of claims 1-9 and 11-26, wherein the ear-wearable device is configured to generate a query for a wearer of the ear-wearable device when an occurrence of head movement consistent with possible head trauma is detected.

11. The ear-wearable device of any of claims 1-10 and 12-26, wherein the ear-wearable device is configured to generate a delayed query for a wearer of the ear-wearable device when an occurrence of head movement consistent with possible head trauma is detected.

12. The ear-wearable device of any of claims 1-11 and 13-26, wherein the ear-wearable device in configured to process signals from the motion sensor to measure at least one of rotational acceleration, rotational velocity, and linear acceleration.

13. The ear-wearable device of any of claims 1-12 and 14-26, wherein the ear-wearable device in configured to process signals from the motion sensor to estimate linear acceleration at a center of gravity of the head of a wearer of the ear-wearable device.

14. The ear-wearable device of any of claims 1-13 and 15-26, wherein the ear-wearable device is configured to monitor signals from the microphone to detect a possible impact event.

15. The ear-wearable device of any of claims 1-14 and 16-26, wherein the ear-wearable device is configured to receive signals regarding detected movement from an accessory device.

16. The ear-wearable device of any of claims 1-15 and 17-26, wherein the accessory device comprises at least one of a second ear-wearable device, a smart phone, and a wearable device.

17. The ear-wearable device of any of claims 1-16 and 18-26, wherein the ear-wearable device is configured to estimate a degree of possible damage to a neck of the ear-wearable device wearer based on a comparison of signals from the motion sensor and signals from the accessory device.

18. The ear-wearable device of any of claims 1-17 and 19-26, wherein the ear-wearable device is configured to a generate an alert if an occurrence of possible head trauma is detected that crosses a threshold value.

19. The ear-wearable device of any of claims 1-18 and 20-26, wherein the ear-wearable device is configured to select an appropriate first target recipient for the alert.

20. The ear-wearable device of any of claims 1-19 and 21-26, wherein the ear-wearable device is configured to select an appropriate first target recipient for the alert based on a predicted severity of possible head trauma.

21. The ear-wearable device of any of claims 1-20 and 22-26, wherein the predicted severity is based on factors including one or more of peak detected acceleration, peak detected velocity, post-incident monitoring data, pre-incident monitoring data, and initial phase detection.

22. The ear-wearable device of any of claims 1-21 and 23-26, wherein post-incident monitoring data includes at least one of microphone signals and motion sensor signals.

23. The ear-wearable device of any of claims 1-22 and 24-26, further comprising:
a physiological sensor, the physiological sensor comprising at least one selected from the group consisting of a blood pressure sensor and a heart rate sensor.

24. The ear-wearable device of any of claims 1-23 and 25-26, wherein the ear-wearable device is configured to stream data based on the signals from at least one of the microphone and the motion sensor after detection an occurrence of head movement consistent with possible head trauma.

25. The ear-wearable device of any of claims 1-24 and 26, wherein the ear-wearable device is configured to evaluate signals from a second sensor to detect a false positive event.

26. The ear-wearable device of any of claims 1-25, wherein the ear-wearable device is a hearing assistance device.

27. An ear-wearable device system comprising:
a first ear-wearable device, the first ear-wearable device comprising
a first control circuit;
a first microphone, wherein the first microphone is in electrical communication with the first control circuit;
a first motion sensor, wherein the first motion sensor is in electrical communication with the first control circuit; and
a first power supply circuit, wherein the first power supply circuit is in electrical communication with the first control circuit;
a second ear-wearable device, the second ear-wearable device comprising
a second control circuit;
a second microphone, wherein the second microphone is in electrical communication with the second control circuit;
a second motion sensor, wherein the second motion sensor is in electrical communication with the second control circuit; and
a second power supply circuit, wherein the second power supply circuit is in electrical communication with the second control circuit;
wherein the ear-wearable device system is configured to process signals from the first motion sensor and the second motion sensor to calculate at least one of rotational acceleration, rotational velocity, and linear acceleration; and
wherein the ear-wearable device system is configured to monitor at least one of rotational acceleration, rotational velocity, and linear acceleration to detect an occurrence of peak acceleration consistent with possible head trauma.

28. The ear-wearable device system of any of claims 27 and 29-52, wherein the ear-wearable device system is configured to change a sampling rate of at least one of the first motion sensor and the second motion sensor if movement is detected consistent with an initial phase of an impact event.

29. The ear-wearable device system of any of claims 27-28 and 30-52, wherein the ear-wearable device system is configured to increase a sampling rate of at least one of the first motion sensor and the second motion sensor if movement is detected consistent with an initial phase of an impact event.

30. The ear-wearable device system of any of claims 27-29 and 31-52, wherein the ear-wearable device system in configured to process signals from at least one of the first motion sensor and the second motion sensor to estimate linear acceleration at a center of gravity of the head of a wearer of the ear-wearable device system.

31. The ear-wearable device system of any of claims 27-30 and 32-52, wherein the ear-wearable device system is configured to record detected occurrences of peak acceleration and/or peak velocity consistent with possible head trauma.

32. The ear-wearable device system of any of claims 27-31 and 33-52, wherein the ear-wearable device system is configured to calculate a cumulative impact index based on the recorded detected occurrences of peak acceleration and/or peak velocity consistent with possible head trauma.

33. The ear-wearable device system of any of claims 27-32 and 34-52, wherein the ear-wearable device system is configured monitor signals from at least one of the first motion sensor and the second motion sensor to detect at least one of dizziness or a fall after detecting an occurrence of head movement consistent with possible head trauma.

34. The ear-wearable device system of any of claims 27-33 and 35-52, wherein the ear-wearable device system is configured to generate an audio warning when an occurrence of peak acceleration and/or velocity consistent with possible head trauma is detected.

35. The ear-wearable device system of any of claims 27-34 and 36-52, wherein the ear-wearable device system is configured to generate an audio warning and send the same to a responsible third party when an occurrence of peak acceleration and/or velocity consistent with possible head trauma is detected.

36. The ear-wearable device system of any of claims 27-35 and 37-52, wherein the ear-wearable device system is configured to generate a query for a wearer of the ear-wearable device system when an occurrence of peak acceleration and/or velocity consistent with possible head trauma is detected.

37. The ear-wearable device system of any of claims 27-36 and 38-52, wherein the ear-wearable device system is configured to generate a delayed query for a wearer of the ear-wearable device system when an occurrence of peak acceleration and/or velocity consistent with possible head trauma is detected.

38. The ear-wearable device system of any of claims 27-37 and 39-52, wherein the ear-wearable device system is configured to monitor signals from at least one of the first microphone and the second microphone to detect a possible impact event.

39. The ear-wearable device system of any of claims 27-38 and 40-52, wherein the ear-wearable device system is configured to receive signals regarding detected movement from an accessory device.

40. The ear-wearable device system of any of claims 27-39 and 41-52, wherein the accessory device comprises at least one of a smart phone and a wearable device.

41. The ear-wearable device system of any of claims 27-40 and 42-52, wherein the ear-wearable device system is configured to estimate a degree of possible damage to a neck of the ear-wearable device system wearer based on a comparison of signals from at least one of the first motion sensor and the second motion sensor and signals from the accessory device.

42. The ear-wearable device system of any of claims 27-41 and 43-52, wherein the ear-wearable device system is configured to a generate an alert if an occurrence of possible head trauma is detected that crosses a threshold value.

43. The ear-wearable device system of any of claims 27-42 and 44-52, wherein the ear-wearable device system is configured to select an appropriate first target recipient for the alert.

44. The ear-wearable device system of any of claims 27-43 and 45-52, wherein the ear-wearable device system is configured to select an appropriate first target recipient for the alert based on a predicted severity of possible head trauma.

45. The ear-wearable device system of any of claims 27-44 and 46-52, wherein the predicted severity is based on factors including one or more of peak detected acceleration, peak detected velocity, post-incident monitoring data, pre-incident monitoring data, and initial phase detection.

46. The ear-wearable device system of any of claims 27-45 and 47-52, wherein post-incident monitoring data includes at least one of microphone signals and motion sensor signals.

47. The ear-wearable device system of any of claims 27-46 and 48-52, further comprising:
a physiological sensor, the physiological sensor comprising at least one selected from the group consisting of a blood pressure sensor and a heart rate sensor.

48. The ear-wearable device system of any of claims 27-47 and 49-52, wherein the ear-wearable device system is configured to stream data based on the signals from at least one of the first microphone, the first motion sensor, the second microphone, and the second motion sensor after detection an occurrence of head movement consistent with possible head trauma.

49. The ear-wearable device system of any of claims 27-48 and 50-52, wherein signals from the first motion sensor or the second motion sensor that cross a threshold value of acceleration and/or velocity are consistent with possible head trauma.

50. The ear-wearable device system of any of claims 27-49 and 51-52, wherein the ear-wearable device system is configured to compare signals from the first motion sensor and the second motion sensor to detect a false positive event.

51. The ear-wearable device system of any of claims 27-50 and 52, wherein the first ear-wearable device is a hearing assistance device.

52. The ear-wearable device system of any of claims 27-51, wherein the second ear-wearable device is a hearing assistance device.

53. A method of tracking head trauma with an ear-wearable device comprising:
evaluating signals from a motion sensor that is part of the ear-wearable device;
calculating at least one of peak rotational acceleration, rotational velocity, and linear acceleration; and
comparing at least one of calculated peak rotational acceleration, rotational velocity, and linear acceleration against one or more threshold values for head trauma.

54. The method of any of claims 53 and 55-74, further comprising recording calculated peak acceleration and/or peak velocity.

55. The method of any of claims 53-54 and 56-74, wherein recording calculated peak acceleration further comprises calculating a cumulative impact index based on the recorded peak acceleration and/or peak velocity values.

56. The method of any of claims 53-55 and 57-74, further comprising changing a sampling rate of the motion sensor if movement is detected consistent with an initial phase of an impact event.

57. The method of any of claims 53-56 and 58-74, further comprising increasing a sampling rate of the motion sensor if movement is detected consistent with an initial phase of an impact event.

58. The method of any of claims 53-57 and 59-74, further comprising monitoring signals from the motion sensor to detect at least one of dizziness or a fall after detecting an occurrence of peak acceleration and/or peak velocity consistent with possible head trauma.

59. The method of any of claims 53-58 and 60-74, further comprising generating an audio warning when an occurrence of peak acceleration and/or peak velocity consistent with possible head trauma is detected.

60. The method of any of claims 53-59 and 61-74, further comprising generating an audio warning and sending the same to a responsible third party when an occurrence of peak acceleration and/or peak velocity consistent with possible head trauma is detected.

61. The method of any of claims 53-60 and 62-74, further comprising generating a query for a wearer of the ear-wearable device when an occurrence of peak acceleration and/or peak velocity consistent with possible head trauma is detected.

62. The method of any of claims 53-61 and 63-74, further comprising generating a delayed query for a wearer of the ear-wearable device when an occurrence of peak acceleration and/or peak velocity consistent with possible head trauma is detected.

63. The method of any of claims 53-62 and 64-74, further comprising estimating linear acceleration at a center of gravity of the head of a wearer of the ear-wearable device.

64. The method of any of claims 53-63 and 65-74, further comprising evaluating signals from a microphone that is part of the ear-wearable device to detect a possible impact event.

65. The method of any of claims 53-64 and 66-74, further comprising receiving signals regarding detected motion from an accessory device.

66. The method of any of claims 53-65 and 67-74, wherein receiving signals regarding detected motion from an accessory device further comprises estimating a degree of possible damage to a neck of the ear-wearable device wearer based on a comparison of signals from the motion sensor and signals from the accessory device.

67. The method of any of claims 53-66 and 68-74, further comprising generating an alert if an occurrence of possible head trauma is detected that crosses a threshold value.

68. The method of any of claims 53-67 and 69-74, further comprising selecting an appropriate first target recipient for the alert.

69. The method of any of claims 53-68 and 70-74, further comprising selecting an appropriate first target recipient for the alert based on a predicted severity of possible head trauma.

70. The method of any of claims 53-69 and 71-74, further comprising predicting severity based on factors including one or more of peak detected acceleration, peak detected velocity, post-incident monitoring data, pre-incident monitoring data, and initial phase detection.

71. The method of any of claims 53-70 and 72-74, wherein post-incident monitoring data includes at least one of microphone signals and motion sensor signals.

72. The method of any of claims 53-71 and 73-74, further comprising evaluating sensor data from at least one sensor selected from the group consisting of a blood pressure sensor and a heart rate sensor.

73. The method of any of claims 53-72 and 74, further comprising streaming data based on signals from at least one of the motion sensor and a microphone after detection an occurrence of head movement consistent with possible head trauma.

74. The method of any of claims 53-73, further comprising evaluating signals of a second sensor to detect a false positive event.
